(19) United States
(12) Patent Application Publication
(10) Pub. No.: US 2005/0249755 A1
(43) Pub. Date: Nov. 10, 2005

(54) Mutant strains of Brucella melitensis and immunogenic compositions

(76) Inventors: Mikeljon Nikolich, Takoma Park, MD (US); David Hoover, Rockville, MD (US); R. Martin Roop II, Greenville, NC (US); Gregory Robertson, Dallas, TX (US)

Correspondence Address: U.S. Army Medical Research and;Materiel Command, 504 Scott Street, Fort Detrick, MD, 21702-5012, US

(21) Appl. No.: 11/071,651

(22) Filed: Mar. 3, 2005

Related U.S. Application Data
(60) Provisional application No. 60/551,011, filed on Mar. 3, 2004.

(51) Int. Cl. A61K 39/10; C12N 15/74; C12N 1/21

(52) U.S. Cl. 424/252.1; 435/252.3; 435/471

(57) ABSTRACT

Live attenuated vaccines against brucellosis are described. New mutant strains of Brucella melitensis have been developed, which are attenuated via deletion of the hfq and/or purEK sites. The purEK deletion site does not include insertion of a kanamycin resistance determinant marker or any other introduced antibiotic resistance marker. The hfq deletion site preferably does not include insertion of a kanamycin resistance determinant marker or any other introduced antibiotic resistance marker.

This application claims priority from U.S. provisional application 60/551,011, filed Mar. 3, 2004, and the contents of U.S. provisional application 60/551,011 are incorporated herein by reference in their entirety.

### BACKGROUND OF THE INVENTION

Brucellosis causes substantial morbidity in humans and exacts a considerable economic toll on both the health care and livestock industries. Of the six recognized species, four (B. melitensis, B. suis, B. abortus, and B. canis) are the classical causes of human disease. B. melitensis causes most human disease and typically causes more severe illness than the other agents. In nature, B. melitensis, B. suis, and B. abortus typically affect goats, swine, and cattle, respectively, causing abortion and/or epididymitis and male infertility. Although many mammals may be infected, most human cases derive from infections in cattle, goats, sheep, and pigs.

The manifestations of infection by Brucella can be reasonably anticipated by considering its pathogenesis. As a facultative intracellular parasite of macrophages and placental trophoblastic cells, Brucella is protected from potential antimicrobial effects of antibody, complement, and therapeutic antimicrobial agents once ingested by its target cells. The outer membrane of the most virulent strains contains long-chain O-polysaccharides. These “smooth” strains frustrate natural host defenses by inhibiting complement deposition to low levels so the organism is phagocytosed but not killed.

Recently, the long-recognized potential use of Brucella as a bioweapon coupled with growing concern over bioterrorism has led to its classification by the United States Centers for Disease Control as a Category B biological threat agent. Elimination of brucellosis in food animals is the referred method to prevent naturally acquired disease in humans, but this approach would not protect against illicit use of the organism as a bioweapon. Development of a human vaccine would be valuable both as a biodefense strategy and as an interim solution for prevention of naturally acquired human disease in situations where economic or sociological factors prevent application of an effective animal disease control program. Indeed, the need to protect occupationally high-risk workers and other susceptible populations has led to a number of efforts at human vaccine development. These efforts have met with variable success, but none has resulted in a well-accepted product. In the near future, however, live attenuated vaccines will be the gold standard for efficacy.

Vaccination has been a key component of animal brucellosis control efforts, but attempts to develop vaccines for humans have met with limited success. Vaccines for livestock are primarily aimed at interruption of transmission. Transmission may occur from mother to offspring by transplacental infection or through milk, or among mature animals venereally or by ingestion or inhalation of bacteria in infected placental tissue after parturition. The primary endpoint of vaccine efficacy in this setting is thus prevention of abortion or newborn infection. Preservation of seronegativity in vaccinated livestock is an important consideration, since it allows detection and elimination of animals that become infected despite vaccination. A less efficacious vaccine that preserves seronegativity may have greater utility than a more effective vaccine that compromises inexpensive identification of infected animals.

Previously developed human vaccines for this disease have been discarded as either unstable, ineffective or unsafe. In humans, in whom person-to-person transmission is of negligible epidemiological importance, the goal of vaccination is prevention or amelioration of disease. Preservation of seronegativity may be less important than safety and efficacy for a human vaccine. In humans, more expensive microbiologic tests and repetitive serologic testing can be used to establish a diagnosis and antimicrobial therapy may be based on a presumptive diagnosis if necessary. For both livestock and humans, vaccines must be safe and elicit a pathogenetically relevant immune response when administered by a feasible route.

Studies have shown that killed vaccines are of limited efficacy and duration of protection, but that live vaccines show promise. Past clinical experience with a strain of B. abortus 19BA and nonhuman primate studies with a strain of B melitensis Rev1, as well as epidemiological data from infections in slaughterhouses, indicate that previous infection or vaccination of humans with live vaccines provides substantial protection against disease.

Live, attenuated vaccines for humans are theoretically attractive because, as indicated by experience in livestock, they are likely to elicit the most solid immunity against infection. A major hurdle to overcome, however, is finding a strain that is both suitably attenuated and sufficiently immunogenic. Although several human vaccines have been tested to date, none is completely satisfactory. Studies have suggested that local reactions may be a limiting factor for vaccines given cutaneously and that killed, whole cell vaccines may have limited efficacy.

At least three live, attenuated vaccines have been tested or fielded in humans. The Brucella strains used included a streptomycin-dependent variant of B. abortus, which had both local and systemic side effects, was ultimately found it to be insufficiently attenuated; infectious doses of live B. melitensis administered by either aerosol or cutaneous vaccination, although neither was desirable due to serious side effects; and another B. abortus strain that resulted in serious side effects, especially in that the range between toxic and effective doses was too narrow. These data suggest that vaccination of humans with live Brucellae leads to protective immunity, but that the vaccines used to date are not sufficiently attenuated. In addition, subcutaneous or cutaneous vaccination may have side effects that can be more severe in previously infected persons.

Current live attenuated vaccine strains used for animals are potentially problematic for humans who work with vaccinated animals because they either can cause disease in humans or carry resistance to antibiotics used for human treatment. Smooth strains currently approved for use in animals are not good candidates for human vaccines because although attenuated, they can still cause disease in humans.

### SUMMARY OF THE INVENTION

The invention is designed to address the lack of a safe vaccine to protect humans against brucellosis. The invention also provides an improvement on the current animal vaccine against brucellosis. The inventors have developed five new strains of Brucella melitensis, and an entirely new approach to Brucella vaccines using these live strains. Specifically, we have developed a vaccine against Brucella melitensis using a live attenuated mutant Brucella melitensis strain.

One main aspect of our invention entails novel strains of mutant Brucella melitensis that can be characterized by having a deletion of the purEK genetic locus, and/or a deletion of the hfq gene. For instance, in a first strain of Brucella melitensis there is a deletion mutation of the purEK operon. As is generally well-known, the purEK operon is required for de novo purine biosynthesis. The sequence and location of the purEK gene within B. melitensis are known. For instance, in the MNP54, MNPH3 and MNPH4 strains of this invention (described below) the purEK deletion is located at positions 309305 through 309532, which were deleted in chromosome I (complete sequence) of the Brucella melitensis strain 16M genome in GenBank. To achieve this deletion, a small 6-bp insertion was introduced into the deletion site, encoding a BglII restriction site (5′-AGACTC-3′). The deletion is located after position 691 in the pMNP54 sequence below, where positions 692-697 of pMNP54 are the inserted BglII site. However, the purEK gene being well characterized, we are not limited to these specific nucleotide positions for the location of the purEK gene.

Importantly, in the Brucella melitensis strains of this invention having a deletion of the purEK gene, there is no introduced antibiotic resistance gene, especially the kanamycin resistance determinant marker gene, inserted at the purEK gene deletion site, as is usually the case—thus, the strain is considered “unmarked” at the purEK site. The kanamycin resistance marker gene codes for kanamycin resistance. In addition, there is no other antibiotic resistance marker inserted in that site. For this particular strain, and the others described below, it is preferable that the Brucella melitensis strain has a 16M genetic background. This background is well known: it is the virulent type strain of B. melitensis in the American Type Culture Collection. An example of this mutant Brucella melitensis strain is the strain designated MNP54, which is on deposit with the American Type Culture Collection. However, for the purposes of this invention, the strains may have any Brucella background.

In a second strain, we have developed a novel mutant Brucella melitensis strain that includes a deletion mutation of the hfq gene. The hfq gene encodes the Brucella homolog of Host Factor I, an RNA-binding protein involved in the regulation of genes involved in stationary phase survival The sequence and location of the purEK gene within B. melitensis are known. For example, the hfq deletion in strains MNPH2 and MNPH4 introduced on pMNH1 (all described below), is located at positions 900261 through 900596, which were deleted in chromosome I (complete sequence) of the Brucella melitensis strain 16M genome in GenBank. The deletion is located after position 1044 in the pMNH1 sequence below. This is a ‘clean’ deletion without any inserted DNA. However, the hfq gene being well characterized, we are not limited to these specific nucleotide positions for the location of the hfq gene.

In this second strain there is a kanamycin resistance determinant marker gene inserted in hfq gene deletion site, so that the strain would be considered “marked” at that site. Preferably, the kanamycin resistance determinant marker gene has the sequence of the Tn903 kanamycin resistance gene, which is well known. An example of this mutant Brucella melitensis strain is the strain designated MNPH1.

In a third strain, we have developed a novel mutant Brucella melitensis strain that also includes a deletion mutation of the hfq gene. In this strain, however, there is no kanamycin resistance marker gene inserted in hfq gene deletion site (or any other introduced antibiotic resistance gene), so that the strain would be considered “unmarked” at that site. This third strain is preferred to the second strain described above, where the hfq site is marked. An example of this mutant Brucella melitensis strain is the strain designated MNPH2, which is on deposit with the American Type Culture Collection.

In a fourth strain, a novel mutant Brucella melitensis strain includes both a deletion mutation of the purEK gene and a deletion mutation of the hfq gene. The purEK deletion site does not have a kanamycin resistance determinant marker gene (or any other introduced antibiotic resistance gene) inserted therein, and is considered “unmarked” at the purEK site. However, there is a kanamycin resistance marker gene inserted in hfq gene deletion site, so that the strain would be considered “marked” at that site. An example of this mutant Brucella melitensis strain is the strain designated MNPH3.

A fifth strain entails a novel mutant Brucella melitensis strain with both a deletion mutation of the purEK gene and a deletion mutation of the hfq gene. Like the fourth strain described just above, the purEK gene site does not have a kanamycin resistance marker gene (or any other introduced antibiotic resistance gene) inserted therein, and is considered “unmarked” at the purEK site. However, there is no kanamycin resistance marker gene (or any other introduced antibiotic resistance gene) inserted in hfq gene deletion site, so that the strain would be considered “unmarked” at that site. An example of this mutant Brucella melitensis strain is the strain designated MNPH4, which is on deposit with the American Type Culture Collection.

All of strains MNP54, MNPH2 and MNPH4 were deposited with the American Type Culture Collection, located at 10801 University Boulevard, Mannassas, Va. 20110-2209.

Another embodiment of our invention entails immunogenic compositions and vaccines comprising at least one of the above-described live attenuated mutant Brucella melitensis strains, and a pharmaceutically acceptable carrier. These strains are useful against brucellosis infection in animals (e.g., wild ruminants, small animals, domestic animals and livestock) and humans. The immunogenic compositions and vaccines comprise at least one of our live mutant Brucella melitensis bacterium strains, all of which have a smooth phenotype, and which are sufficiently attenuated or otherwise inactivated that upon exposure to a mammal the strain will not exhibit full virulence of non-attenuated/non-inactivated Brucella. For animal vaccines the strain must be attenuated or inactivated enough to be safely used in animals, and for human vaccines the strain must be safe enough to be used in humans. Attenuation is accomplished by either a single mutation or a double mutation. Specifically, the Brucella melitensis strain may be singly attenuated with either the hfq deletion or the purEK deletion, or may be doubly attenuated with both the hfq deletion and the purEK deletion. If attenuated via hfq deletion, the deletion site may be marked with a kanamycin resistance marker, or may contain no kanamycin resistance marker. If attenuated via purE deletion, the deletion site contains no kanamycin resistance marker.

Our invention encompasses methods for inducing protective immunity to brucellosis in a mammal, comprising the step of administering to a mammal a vaccine comprising one or more of the live attenuated mutant Brucella melitensis strains described above, with a pharmaceutically acceptable carrier. For instance, we contemplate vaccines for small ruminants such as goats and sheep in areas where B. melitensis is endemic, useful in particular for stopping the spread of brucellosis amongst ruminants that come into contact with and potentially infect humans.

### BRIEF DESCRIPTION OF THE FIGURES

FIG. 1 shows W9901 bacteremia following oral gavage of Rhesus macaques with 1×10¹² CFU/ml of Brucella melitensis Strain 16M.

FIG. 2 shows W9901 tissue recovery of Brucella melitensis Strain 16M at four weeks from Rhesus macaques given 1×10¹² CFU by gavage.

FIG. 3 shows experiment number IO05-02 spleen area by ultrasound for adult male Rhesus macaques given 1×10¹² (Group 1) or 1×10¹¹ CFU (Group 2) of Brucella melitensis vaccine candidate MNPH1 by gavage.

FIG. 4 shows experiment number IO05-02 tissue recovery of Brucella melitensis Strain MNPH1 from adult male Rhesus macaques given either 1×10¹² or 1×10¹¹ CFU by gavage.

FIG. 5 shows experiment number IO10-02 body weight as percent of baseline measurement in adult male Rhesus macaques vaccinated with 1×10¹¹ CFU of vaccine candidate MNPH1 and subsequently challenged by the conjunctival route with 1×10⁷ CFU of Brucella melitensis Strain 16M.

FIG. 6 shows experiment number IO10-02 spleen area by ultrasound in adult Rhesus macaques vaccinated with 1×10¹¹ CFU of vaccine candidate MNPH1 given by gavage and challenged via the conjunctival route with 1×10⁷ CFU of Brucella melitensis Strain 16M.

FIG. 7 shows experiment number 1005-02 anti-Brucella melitensis LPS by ELISA for adult Rhesus macaques given vaccine candidate MNPH1 by gavage.

FIG. 8 A, B and C show experiment number IO05-02 febrile data of vaccine candidate MNPH1 given to adult male Rhesus macaques. None of the vaccinated macaques became febrile.

FIG. 9 shows experiment number IO10-02 bacteremia of vaccine candidate MNPH1 given to adult male Rhesus macaques (N=8) at a dose of 1×10¹¹ CFU by gavage.

FIG. 10 shows experiment number IO10-02 bacteremia in adult Rhesus macaques of Brucella melitensis Strain 16M given via conjunctival challenge at a dose of 1×10⁷ CFU.

FIG. 11 shows the hfq allelic exchange knockout plasmid pFR4kn, and the progression of deletion of the hfq gene and the insert of the kanamycin cassette in construction of strains such as MNPH1.

FIGS. 12-15 show that strains MNPH1, MNPH2, MNPH3 and MNP54 were all reduced in their ability, relative to virulent strain 16M, to survive in these human macrophages.

FIG. 12 shows Brucella vaccine strains in human monocyte-derived macrophage experiments. Strains tested were MNPH1 and MNP54 relative to attenuated strain WR201 and virulent strain 16M. Strain 16M is the wild type control; MNP54 and WR201 are both attenuated purEK deletion strains. This Figure shows intracellular survival of MNPH1 under starvation, hydrogen peroxide and low pH conditions found in cultured Human macrophages derived from peripheral blood monocytes (monocyte-derived macrophages or MDMs). This indicates that both strain MNP54 and MNPH1 were attenuated and both are quite similar to WR201 in their ability to survice in human macrophages.

FIG. 13 shows Brucella vaccine strains in human monocyte-derived macrophage experiments. Strains tested were MNPH1 and MNP54 relative to virulent strain 16M. Strain 16M is wild type control; MNP54 is an attenuated purEK deletion strain. MNPB3 is a putative attenuated strain. Because of fungal contamination, accurate counts at some timepoints for MNPH1 and MNP54 could not be measured. MNPB3 is another attenuated strain.

FIG. 14 s shows the results of experiments that indicate that unmarked hfq strain MNPH2 was better able to survive in macrophages than kanamycin resistant strain MNPH. MNPH2 did appear significantly attenuated in this model relative to 16M. Strain 16M/6Y is the wild type control. MNPH2 and MNPH3 are putative attenuated strains. Strains tested were MNPH1, MNPH2 and MNPH3 relative to virulent strain 16M (here bearing a GFP plasmid). In this experiment strains MNPH1 and MNPH3 were not detected at later timepoints. Monocytes were cultured for 20 days prior to infection.

FIG. 15 shows Brucella vaccine strains in human monocyte-derived macrophage experiments. Strain MNPH3 was tested relative to attenuated strain WR201 and highly attenuated wboA purEK dual mutant strain WRRP1.

FIGS. 16 and 17 show that in the mouse model dual mutant strain MNPH3 is more attenuated than strain MNPH1.

FIG. 16 shows Brucella vaccine strain MNPH3 in BALB/c mice after 10¹¹ oral dose. The graph shows the persistence of this strain in mouse spleens.

FIG. 17 shows Brucella vaccine strain MNPH3 in BALB/c mice after 10¹¹ oral dose. The graph shows the percent mice infected in the spleen.

FIG. 18 is a map of pMN54.

FIG. 19 is a map of pMNH3 (7.5 kb)

FIG. 20 shows the results of experiments that indicated that putative B. melitensis hfq mutant strain GR140, which contains a single deletion at the hfq site and has a kanamycin resistance marker inserted therein, was better able to survive in human macrophages than hfq strain MNPH1. This is consistent with what was observed in later experiments. Strain 16M is the wild type control; MNP54 is an attenuated purEK deletion strain. GR140 is a putative attenuated strain.

FIG. 21 is a map of pMNH1.

FIG. 22 shows the results of experiments that indicate that unmarked hfq strain MNPH2 was better able to survive in macrophages than kanamycin resistant strain MNPH. MNPH2 did appear significantly attenuated in this model relative to 16M. Strain 16M is the wild type control. GR140 and MNPB3 are putative attenuated strains. Monocytes in this experiment were cultured for 19 days prior to infection.

FIG. 23 shows the results of experiments that indicated that putative B. melitensis hfq mutant strain GR140, which contains a single deletion at the hfq site and has a kanamycin resistance marker inserted therein, was better able to survive in human macrophages than hfq strain MNPH1. This is consistent with what was observed in later experiments. Strain 16M is the wild type control; MNP54 is an attenuated purEK deletion strain. MNPH2 is a putative attenuated strain.

FIG. 24 the results of experiments that indicated that putative B. melitensis hfq mutant strain GR140, which contains a single deletion at the hfq site and has a kanamycin resistance marker inserted therein, was better able to survive in human macrophages than hfq strain MNPH1. This is consistent with what was observed in later experiments. In FIG. 24A strain 16M is the wild type control. GR140 and MNPB3 are putative attenuated strains. Monocytes were cultured for one week before infection. In FIG. 24B, the strain 16M is the wild type control. GR140 and MNPB3 are putative attenuated strains. Monocytes were cultured for two weeks before infection.

### DETAILED DESCRIPTION OF THE PREFERRED EMBODIMENTS

The invention described here includes a series of attenuated smooth phenotype Brucella melitensis strains with one or two defined nonreverting genetic mutations intended for use as live vaccines to protect humans and other mammals against brucellosis. The inventors have developed live smooth vaccines and immunogenic compositions that are safe and efficacious for veterinary and human vaccine purposes.

In developing these strains, the inventors sought to create lines containing multiple attenuating mutations, to lessen the possibility of reversion to virulence. The inventors speculated that strains carrying multiple, diverse attenuating mutations may demonstrate various degrees of attenuation, making it possible to select vaccine candidates demonstrating the desired balance between rapid clearance from the host and induction of protective immune responses. Finding the right balance is critical in the development of live, attenuated vaccines as it is well known that strains that are cleared from the host too rapidly may be inefficient in eliciting protective immune response. On the other hand, strains which retain considerable residual virulence may produce overt disease in immunocompromised hosts.

As is described here, the inventors constructed such Brucella melitensis strains containing single or double mutations of the hfq and purE genes. In the examples section below, the construction, verification and testing of these strains is described where the particular strains created and tested are designated MNP54, MNPH1, MNPH2, MNPH3 and MNPH4, depending on whether the strain is singly or doubly mutated and whether the hfq deletion site is marked or unmarked with a kanamycin resistance determinant marker. As someone having ordinary skill in this art would readily appreciate, we do not intend by these examples and these designations to limit the scope of our invention to these specific strains as designated. That is, we do not limit our invention solely to the specific genetic sequences of these strains. While these specific strains are within the scope of our invention, and are examples of the mutant strains we contemplate here, our invention is significantly broader in scope. For instance, our invention includes B. melitensis strains that are mutated by deletion of the purEK gene, where there is not kanamycin resistance determinant marker inserted in the purEK gene deletion site. The strain designated MNP54 is but one example of a strain that meets this criteria.

With particular regard to the MNP54 strain, this strain was a variant of strain WR201 (described in U.S. Pat. No. 5,939,075), and was constructed using allelic exchange with the Bacillus subtilus levansucrase-encoding sacB as a counterselectable marker. Using this approach we also developed a dual mutant which has a secondary deletion mutation in hfq (in the examples it is designated MNPH3). This purEK hfq double mutant appeared to be at least as impaired in its ability to replicate in human macrophages as either strain MNP54 or strain MNPH1 (marked hfq deletion). It is generally believed to be a safer strain for use in vaccines, however, due to the second, “backup” mutation—which is an advantage in seeking FDA approval.

Another strain, was created by making a 336 bp deletion, including 173 bp of the hfq gene, from virulent B. melitensis 16M and inserting a kanamycin resistance cassette. In the example section it is designated MNPH1. The hfq gene product regulates responses of Brucella to stationary phase conditions. As shown in the Examples section, MNPH1 does not replicate in human monocyte-derived macrophages, while 16M increases 2 logs over 48 hr of culture. MNPH1 does not have the ability to multiply inside human macrophage the way the parent strain does, which indicates reduced virulence.

Any of these strains may be used as either human or animal vaccines, either alive, somehow made inactive or nonviable, or killed. Any of the strains herein may be effective as here constituted or may be manipulated to be made even more safe or more immunogenic. Adjuvants may be added or antigens may be added or boosted to increase the effectiveness of these vaccines. Any of these strains may be used as a platform for the intracellular delivery of antigenic material from other disease agents in a vaccine formulation. Any of these strains may be used as an adjuvant for another vaccine or a stimulator of nonspecific immunity in the recipient. Any of these strains may be used as a host cell for the expression, production and purification of any native antigen or other native material. Any of these strains could be used as a Brucella antigen.

Our Brucella strains are derived from Brucella melitensis. This is advantageous over other Brucella strains such as Brucella abortus because B. melitensis is the species/biovar that causes the most human disease in the Brucella group of pathogesn. Also, B. melitensis is better suited for use in goat vaccines, since goats are a natural host. More researchers are adverse to working with B. melitensis because it is significantly more virulent in humans that B. abortus.

One novel aspect of our invention is the removal of antibiotic resistance genes from the hfq deletion site (for instance, as in strains MNPH2 and MNPH4). Similarly, another novel aspect is the removal of antibiotic resistance genes from the purEK deletion site (for instance, as in strains MNP54, MNPH3 and MNPH4). Removal of these markers was technically difficult. The sacB system is tricky in general, but particularly when one is working with an organism like B. melitensis where recombinant rates can already be very low. It also lends an advantage to our strains by making them safer to use in animal and human vaccines. For instance, a B. melitensis purEK mutant strain, WR201 (also known as deltapurE201) was previously constructed as per U.S. Pat. No. 5,939,075, which strain contained a kanamycin resistance marker in the purEK deletion site. That marker is potentially problematic in that such antibiotic resistance markers are viewed as less safe and therefore undesirable as vaccines. FDA approval for the WR201 strain is questionable for this reason.

Another strains, GR140, described in Roop et al. 2003, is also considered less safe than the strains of our invention, especially MNPH1. MNPH1 clears from humn macrophages faster than GR140, and therefore is more attenuated and deemed the safer strain.

Another novel aspect of our strains is the combination of purEK and hfq mutations in a Brucella vaccine strain (for instance, as in strains MNPH3 and MNPH4). The combination of multiple attenuating mutations in a live vaccine strain introduces an increased degree of safety for vaccinees over single mutation strains. Here again is another advantage.

As demonstrated in more details in the examples below, the ability of these strains to survive within human host cells was assessed by infecting human monocyte-derived macrophages. The intracellular survival of these new live vaccine candidates relative to virulent B. melitensis strain 16M and attenuated B. melitensis vaccine strain WR201 was compared in an in vitro assay. All of the exemplary strains were reduced in their ability, relative to virulent strain 16M, to survive in these human macrophages. Strains MNPH1 (hfq) and MNPH3 (purEK/hfq) appeared to survive at levels comparable to purEK mutants MNP54 and WR201. Strain MNPH2 (unmarked hfq) appeared to persist in slightly higher numbers. In both mouse and nonhuman primate models strain MNPH1 was significantly attenuated. When strain MNPH1 was given orally to mice it provided significant protection against intranasal challenge with virulent Brucella. In a mouse model it was shown that dual mutant strain MNPH3 is more attenuated that strain MNPH 1.

In in vitro studies, hfq mutants of B. abortus are more sensitive than parents to starvation, hydrogen peroxide, and low pH, conditions encountered by the bacterium en route to or inside its intramacrophage niche, an acidified phagosomal compartment. One vaccine candidate, B. melitensis MNPH1, does not replicate in human monocyte-derived macrophages, while virulent B. melitensis 16M increases 2 logs over 48 hr of culture. When given orally to mice or Rhesus macaques, MNPH1 disseminates through the bloodstream and localizes in lung, liver, spleen, and lymph nodes. In mice, bacteria are nearly completely eliminated from these organs by 8 weeks and are completely eliminated by 12 weeks. In monkeys, MNPH1 is still present in the lymph nodes 8 weeks after inoculation of 10¹¹ CFU. Fifteen weeks after oral inoculation with MNPH1 and 6 weeks after challenge with 16M, only one colony of vaccine strain was recovered from one lymph node of one of four monkeys. No histopathological abnormalities were attributed to infection with MNPH1 in mice or monkeys. In contrast, oral infection of mice or monkeys with virulent parent strain 16M leads to greater intensity of infection in lymphoid organs. In addition, 16M infect nonlymphoid organisms, accompanied by marked histopathological abnormalities. In addition, 16M persist at high levels in tissues of mice for at least 16 weeks. MNPH1 is shed in murine feces for up to 9 days after an oral dose of 10¹¹ CFU. In monkeys, MNPH1 is shed in feces for up to 3 days, depending on dose. In monkeys, intensity of disseminated infection by MNPH1 is dose-related. Mice and monkeys develop serum anti-LPS IgG after inoculation with MNPH1. Culture of MNPH1-immune mouse spleens in the presence of Brucella antigens leads to production of IFN-□ and IL-2 in culture supernatant fluids. Animals inoculated orally with MNPH1 are protected from mucosal challenge with virulent B. melitensis. In mice, protection against intranasal challenge is manifested by reduced percentage of animals with dissemination to spleen or liver. In monkeys, protection is manifested by reduced intensity and frequency of bacteremia, absence of fever, absence of infection of organs outside the mononuclear phagocyte system and reduced intensity of infection of the mononuclear phagocyte system by the challenge organism.

Thus, introduction of the hfq deletion alone provides significant safety improvements in the B. melitensis strains, for use as human and animal vaccines. The addition of the purEK mutation only improves the safety further. In testing, the unmarked hfq strain in MNPH4 was different from the marked MNPH1, indicating that MNPH4 is less impaired and more immunologically protective.

These studies indicate that inoculation of mice and nonhuman primates with MNPH1 and the other strains leads to short-term fecal shedding, brief bacteremia, and self-limited infection of the mononuclear phagocyte system. Fecal shedding and intensity of mononuclear phagocyte system infection are reduced with a lower dose. MNPH1 appears sufficiently safe for use in humans and the other strains. While fully virulent strains of Brucella may cause serious infection of the heart valves and central nervous system, these complications are uncommon. Other complications of infection with virulent strains include infection of the musculoskeletal and mononuclear phagocyte systems, male genitourinary tract, and potentially abortion in females. The risk of these complications would be minimized by careful clinical monitoring and treatment with clinically effective antibiotics at the conclusion of the study and earlier if disease occurs.

One should note that infection of these same organs and fatal illness occur with virulent Mycobacterium tuberculosis and Salmonella. Nevertheless, live, attenuated vaccines for these intracellular bacterial pathogens have been developed and are used clinically for prevention of human mycobacterial infection (Mycobacterium bovis Bacillus Calmette-Guerin) and typhoid fever (Salmonella typhi Ty21a). In addition, the level of attenuation of MNPH1 demonstrated in these animal studies compares favorably to attenuation of other live vaccines in similar models. Intraperitoneal administration of 1 CFU of Francisella tularensis LVS strain, for example, is lethal for 50% of mice, while skin LD₅₀ is >10⁴ CFU (Elkins et al., 1002, “Introduction of Francisella tularensis at skin sites induces resistance to infection and generation of protective immunity”, Microb. Pathog. 13:417-21). A dose of 10⁵ CFU of LVS administered intracutaneously to monkeys (Macaca irus) does not cause detectable bacteremia, but leads to infection in the draining lymph nodes for at least 28 days, infection of liver for at least 10 days, and infection of spleen for at least 14 days (Eigelsbach et al., 1962, “Live tularemia vaccine. I. Host-parasite relationship in monkeys vaccinated intracutaneously or aerogenically”, J. Bacteriol. 84:1020-1027). The successful development of these vaccines provides precedent for testing of a live vaccine for brucellosis.

In terms of a human use, the vaccines could be used in deployable troops in a 1 or 2-dose oral regimen to protect against Brucella melitensis. Because of known cross-protection, the vaccines could be useful to protect against Brucella abortus and Brucella suis.

To that end, our novel Brucella strains would find ready application in several arenas. For example, since Brucella is a potential biowarfare agent and thus a threat to U.S. military personnel, the Department of Defense would be a potential user of this technology, especially as regards vaccines against Brucella. Similarly, the Department of Homeland Security may also be interested in these vaccines against a Brucella bioterror threat, which may be useful against potential bioterrorism aimed at agriculture and populace.

Since Brucella causes disease in a variety of livestock, the U.S. Department of Agriculture would also have potential uses for this technology, since it offers the potential of single vaccination versus multiple animal diseases. Furthermore, pharmaceutical and agribusiness firms would be interested in using our vaccines to create commercial vaccines for people and livestock. These smooth Brucella strains are more highly attenuated than the Rev1 strain currently administered to goats as a vaccine. The animals vaccines may be used in small ruminants to protect against transmission among each other, as well as to humans coming into contact.

### EXAMPLES

The invention in various embodiments and stages is further described in the following non-limiting examples.

### Example 1

This example gives details of the genetic construction and verification of the five new strains.

Strains and growth conditions. All Brucella strains were grown in Brucella broth or on Brucella agar or M9 minimal agar. Brucella melitensis strains 16M (wild type), WR201 (ΔpurE201 (see Drazek, E., et al., Deletion of purE attenuates Brucella melitensis 16M for growth in human monocyte-derived macrophages. Infect Immun, 1995. 63(9): p. 3297-3301)), MNPH1, MNPH2 and MNPH3 were used to infect monocyte-derived macrophages (MDMs) and mice. Stocks of exponential-phase cultures frozen in 20% glycerol were thawed and used to inoculate. Broth cultures were shaken at 37° C. for 22 to 26 hours until an absorbance measurement at 600 nm of 0.4 to 0.8 by spectrophotometer (Spectronic Instruments, Rochester, N.Y.) was obtained. Bacterial cells were then harvested by centrifuge, washed in sterile 0.9% NaCl, and adjusted to the correct dilution in the same. Turbidity was used to estimate viable bacterial counts and t thus determine the appropriate dose for infection. Actual colony-forming units (CFUs) in the inocula were determined by serial dilution and plating on Brucella agar. Plate counts were made after seven days of incubation at 37° C., unless otherwise noted.

Animals. BALB/C mice were obtained from Harlan Sprague Dawley, Frederick, Md., and were used at eight to twelve weeks of age. Mice were Brucella-free and were kept in a Biosafety level three (BSL-3) facility. Studies were performed according to Walter Reed Army Institute of Research and all other regulations and guidelines regarding the use of laboratory animals in research.

Construction of B. melitensis deletion mutants. A deletion mutant of B. melitensis strain 16M designated MNPH1 was constructed by replacing its genomic hfq locus with the deleted and kanamycin-resistance marked allele on pGR4kan by homologous recombination as previously described (See Robertson, G. T. and R. M. R. II, The Brucella abortus host factor I (HF-I) protein contributes to stress resistance during stationary phase and is a major determinant of virulence in mice. Mol Microbiol, 1999. 34(4): p. 690-700.) Verification of the replacement of the hfq locus in MNPH1 with the deleted allele with kanamycin cassette insert was done using oligonucleotide primers hfq200-up (5′ ACG CCT CGC CCG CAA ATA C 3′) (SEQ ID NO:1) and hfq200-lo (5'CCT CGC CTT CAA ACA TCT G 3′) (SEQ ID NO:2).

An allelic exchange approach was employed to remove the kanamycin resistance marker from the hfq deletion in strain MNPH1 using sacB as counterselection for the genereplacement vector. A gene replacement plasmid for this purpose was constructed by first removing the kanamycin resistance marker inserted in the hfq deletion on pGR4kan by digesting with BstXI and SphI, blunting the ends with T4 DNA polymerase and ligating to create pGR4kanΔk. The sacB marker on pEX100T (See Schweizer, H. P. and T. T. Hoang, An improved system for gene replacement and xy1E fusion analysis in Pseudomonas aeruginosa*1. Gene, 1995. 158(1): p. 15-22. was amplified by PCR with oligonucleotide primers sacU (5′ ATA AAA ATA GGC GTA TCA CGA G 3′) (SEQ ID NO:3) and sacL (5′ AAA GAG GAA AAT AGA CCA GTT G 3′) (SEQ ID NO:4) and inserted into pCR2.1 using the TOPO TA Cloning kit (Invitrogen, Carlsbad, Calif.) to make pCRsac2.

The region on pGR4kanΔk containing the unmarked deleted hfq locus was then subcloned on a SstI-HindIII fragment into pCRsac2 opened with SstI and HindIII to make the gene replacement plasmid pMNH1. Strain MNPH1 was electroporated with pMNH1. Ampicillin resistant electroporants were incubated in Brucella broth without selection for 48 h and then plated on sucrose medium (w/v, 1% tryptone, 0.5% yeast extract, 5% sucrose) to select for the sucrose resistant sacB-phenotype. Half of the sucrose resistant colonies so isolated were sensitive to kanamycin. Removal of the kanamycin cassette from the hfq deletion site in these kanamycin sensitive derivatives was confirmed by PCR using oligonucleotide primers hfq200-up and hfq200-lo. The deletion was also confirmed by Southern blot; genomic DNA was digested with EcoRI and probed with the 1.6 kb SstI-HindIII fragment of pGR4kanΔk. An isolate so confirmed was designated strain MNPH2.

In order to construct a dual deletion mutant of B. melitensis in both purEK and hfq loci, first an unmarked purEK deletion mutant of B. melitensis strain 16M was constructed. A region of purE197 containing the purEK locus cloned from the genome of B. melitensis strain 16M with the 3′ 195 bp of purE, the intergenic sequence and the 5′ 17 bp of purK deleted (see Drazek, E., et al., Deletion of purE attenuates Brucella melitensis 16M for growth in human monocyte-derived macrophages. Infect Immun, 1995. 63(9): p. 3297-3301) was amplified by Polymerase Chain Reaction using oligonucleotide primers puramp-U (5′ GAC TAG TAG GCT TTA CAC TTT ATG CTT 3′) (SEQ ID NO:5) and puramp-L (5′ CAC TAG TAT CTT TTC TAC GGG GTC TGA 3′) (SEQ ID NO:6). The SpeI tails of the amplicon were then digested (Promega, Madison, Wis.) and the fragment was then inserted into the SpeI site of pJQ200KS (See Quandt, J., and M. F. Hynes, Versatile suicide vectors which allow direct selection for gene replacement in Gram-negative bacteria. Gene, 1993. 127: p. 15-21) using T4 DNA Ligase (Life Technologies, Rockville, Md.). The copy of the Bacillus subtilus sacB on pJQ200KS encoding levansucrase was to be used as a conditional counterselection for exclusion of the vector DNA from the B. melitensis strain 16M genome after integration by homologous recombination. The resulting sacB ΔpurEK allelic exchange plasmid, pMNP54, was used to replace the intact purEK operon in the genome of B. melitensis 16M with a deleted allele without an inserted antibiotic resistance determinants.

Electroporants with chromosomally integrated pMNP54 were recovered on Brucella agar containing 50 μg/ml ampicillin. Ampicillin-resistant colonies were incubated in Brucella broth without selection for 48 h and then plated on sucrose medium (w/v, 1% tryptone, 0.5% yeast extract, 5% sucrose) to select for resolution of the integrated knockout vector via loss of sacB, which confers sucrose sensitivity on Brucella. Sucrose-resistant, ampicillin-sensitive colonies derived in this fashion were screened for purine auxotrophy on M9 agar with and without purine supplementation. An unmarked purine auxotrophic derivative of B. melitensis strain 16M thus selected was then genotype confirmed by PCR using oligonucleotide primers to amplify the majority of the purEK operon, puEup1.1096 (5'CAC CAT GCA GCC GAC ACA 3′) (SEQ ID NO:7) and purKdn1.1096 (5'CGC GCC GCA GAT TCA GGG 3′) (SEQ ID NO:8), and each of these respectively in combination with oligonucleotides designed to prime from within the deleted region, EKdeltaD.1096 (5′ GAG TGC CGA CGG GAA TAC 3′) (SEQ ID NO:9) and EKdeltaU.1096 (5′ GAT CCG GCG AGG TAG AAA 3′) (SEQ ID NO:10). Southern blots including genomic digestions with BglII, ClaI and EcoRI were probed with a puEup1.1096-purKdn1.1096 strain 16M amplicon and also confirmed the presence of a deleted allele in place of the purEK locus. The confirmed mutant was designated strain MNP54.

Strain MNP54 was used as the parental strain for the introduction of a second attenuating mutation in hfq to create a purEK hfq dual deletion mutant of B. melitensis. The hfq knockout plasmid pGR4kan was used to replace the intact hfq allele in MNP54 with an allele containing a 337 bp deletion (total) in hfq with a kanamycin resistance cassette insert as described above for construction of strain MNPH1. The resulting strain, B. melitensis MNPH3, was kanamycin resistant and confirmed by PCR with primer sets hfq200-up and hfq200-lo to have the predicted deletion in hfq. Strain MNPH3 was also confirmed by PCR using oligonucleotide primers purEK2-up (5'CCG AAA AGC CAA GCA GGA AAG 3′) (SEQ ID NO:11) and purEK2-dn (5′ GCC GGG CGT CAT AAA AAC AGG 3′) (SEQ ID NO:12) to retain the unmarked deletion in purEK.

An unmarked hfq deletion was also introduced into strain MNP54 create a purEK hfq dual deletion mutant of B. melitensis without any introduced antibiotic resistance markers. The knockout plasmid containing the unmarked hfq deletion, pMNH1, was used to replace the intact hfq allele in MNP54 with an allele containing a 236 bp deletion in hfq as described above. The resulting strain, designated MNPH4, was confirmed by PCR with primer sets hfq200-up and hfq200-lo to have the predicted deletion in hfq.

To move plasmid DNA into B. melitensis strains, the recipient strain was grown for 24 or more hours in YENB (see Sharma, R. C., and R. T. Schimke, Preparation of electro-competent E. coli using salt-free growth medium. BioTechniques, 1996. 20: p. 42-44.) pelleted, washed in ½ volume cold 10% glycerol and resuspended in 1/10 volume of the same. In cuvettes 1 μg of plasmid DNA was added to 100 μl of the electrocompetent bacteria and then electroporated at 2.5 kV, 25 μF and 600 Ω. One ml of SOC (Life Technologies, Rockville, Md.) was added and the mix was incubated at 37° C. with shaking for two hours. The electroporation mix was then plated on Brucella agar containing the appropriate selective antibiotic (50 μg/ml ampicillin or 50 μg/ml kanamycin).

Putative mutants were verified by genotype using PCR and Southern hybridization. Chromosomal DNA was isolated using an adaptation of the Marmur procedure (see Marmur, J., A procedure for the isolation of deoxyribonucleic acid from microorganisms. J Mol Biol, 1961. 3: p. 208-218.). Polymerase chain reaction amplification of sequences was done by standard methods using Amplitaq Gold (Perkin Elmer), Platinum Taq or Platinum Taq HiFi (Life Technologies, Rockville, Md.). For Southern hybridizations, chromosomal DNA preparations were digested with restriction enzymes (Life Technologies, Rockville, Md., and New England Biolabs, Beverly, Mass.) electroporated on a 1% agarose slab gel in TAE buffer, and transferred in a positive pressure cell (Stratagene, LaJolla, Calif.) to a Nytran membrane (Schleicher and Schuell, Keene, N H). DNA probes were directly labeled nonisotopically using ECL and AlkPhos chemistry and chemiluminescent detection (Amersham Life Sciences, Birminghamshire, England). Procedures not specified above were done by standard methodology. (See Ausubel, ed. Current protocols in molecular biology. 1994, Greene Publishing Associates: New York, N.Y.; and Sambrook, ed. Molecular cloning, a laboratory manual. 2nd ed. 1989: Cold Spring Harbor, N.Y.)

Infection of human monocyte-derived macrophages. Mononuclear cells obtained by centrifuging leukopaks from normal donors over lymphocyte separation medium (Organon Teknika, Durham, N.C.). Counterflow centrifugal elutriation was then used to further purify the monocytes (see Wahl, L. M., Isolation of human mononuclear cell subsets by counter flow centrifugal elutriation (CCE). Cell Immunol, 1984. 85: p. 373-383), resulting in preparations with greater than 95% viability (estimated by trypan exclusion) and <10% lymphocytes. Monocytes were suspended in MDM medium [RPMI 1640 medium (Life Technologies, Inc., Gaithersburg, Md.) with 10% heat-inactivated human serum (Sigma Chemical Co., St. Louis, Mo.), 2 mM L-glutamine and 10 ng/ml of recombinant human macrophage colony stimulating factor (Jay Stoudemire, Genetics Institute, Boston, Mass.)]. This suspension was added to wells in a sterile tissue culture plate and 10⁵ cells per well were cultured as adherent monolayers at 37° C. in a 5% CO₂ incubator. On the fourth day and again on the seventh day, half of the medium was removed and replaced with fresh medium. On the eighth day of the experiment, the media in the MDM culture wells was removed and replaced with the medium described above, except with 10% unheated normal serum in place of Sigma serum. Brucella strains were grown as stated above. Broth media did not contain antibiotics to avoid carryover; cultures used to infect were plated selective and nonselective agar to verify that antibiotic resistance was maintained uniformly in strains that bear these markers. After the saline wash, Brucellae were resuspended to 2×10⁸ CFU/ml in saline and added to MDM wells at a multiplicity of infection of 10:1. Infections proceeded for 60 min. at 37° C., then monolayers were washed three times with RPMI with 10% Sigma serum and 2 mM L-glutamine. MDM medium with 1 μg/ml gentamycin was then added to wells and plates were incubated at 37° C. in a 5% CO₂ incubator. At various timepoints plates were removed and MDM monolayers were washed as before, and then lysed by adding 0.1% Triton X-100 and mixing vigorously. Lysates were diluted serially in sterile saline, and these dilutions were plated on Brucella agar to obtain viable counts. Presented here are mean values from three MDM wells, dilutions from each plated in duplicate. The data shown are representative of at least four different experiments.

Survival in mice. BALB/c mice were inoculated via the oral route with 1011 CFU of Brucella vaccine strains. Brucella strains were prepared by growing them overnight in Brucella broth in flasks at 37° C. with shaking after inoculation from frozen secondary research stocks. Bacteria were grown for 3 days on Brucella agar plates. Bacterial lawns were then scraped from the agar using sterile technique, washed twice in 0.9% NaCl (saline). Bacteria were then resuspended in saline at a concentration of 5×10¹¹ CFU/ml based on turbidity measured at an absorbance setting of 600 nm. Using a syringe fitted with a 20 gauge disposable feeding needle, 0.2 ml of 2.5% sodium bicarbonate was administered to mice via the oral route; 15 minutes later 0.2 ml of adjusted Brucella strain suspension was introduced by the same method. Animals were not anesthetized during immunization. Actual viable inocula were determined by dilution and plating on Brucella agar. At selected times from 1 day to 12 weeks after inoculation, mice were euthanized by CO₂ asphyxiation and organs were harvested to determine the extent of infection and eventual clearance. Organs were homogenized, diluted and plated on Brucella agar containing 25 μg/ml bacitracin and 5 μg/ml polymixin B (also with 50 μg/ml kanamycin when appropriate) to determine viable counts of Brucella per organ. Plates were incubated at 37° C. for five days before enumeration. Serum was also collected from each mouse. Spleen cells were cultured at 4 weeks post infection. The first two experiments used female mice; the last three examined male mice, since male genitalia are a target of Brucella infection.

Oral immunization and intranasal challenge of mice. Brucella strains were grown in Brucella broth and then on agar plates, washed, suspended and administered orally as described above. Intranasal challenge of immunized mice with virulent B. melitensis strain 16M was performed four weeks after vaccination as previously described (see Hoover, D. L., et al., Protection of Mice against Brucellosis by Vaccination with Brucella melitensis WR201(16MDelta purEK). Infect Immun, 1999. 67(11): p. 5877-5884). A suspension of strain 16M containing 10⁴ CFU was delivered dropwise into the external nares of mice anesthetized with xylazine and ketamine. At selected times after challenge mice were euthanized and organs removed, homogenized, suspended in saline and diluted and plated on Brucella agar containing 25 μg/ml bacitracin and 5 μg/ml polymixin B.

### Example 2

### Mouse Studies

This examples provides details of the construction and verification of strain MNPH1 and testing of its attenuation and efficacy in animal models.

For most studies, organisms are grown in BBL Brucella broth and shaken at 37° C., and maintained in cryovials with 0.5 ml sterile glycerol at −70° C. For an ongoing study, bacteria were prepared by fermentation in trypticase soy broth (TSB) and frozen in rubber-stoppered glass vials in 5% sucrose.

The mouse studies were designed to demonstrate that MNPH1 is highly attenuated for infection in BALB/c mice. This is achieved through examining the dissemination and persistence of infection of various organs when compared to B. melitensis 16M, the wild-type parent. In addition, the studies aimed to demonstrate the immunogenicity and efficacy of MNPH1 immunization in mice when subsequently challenged with B. melitensis 16M.

Attenuation of MNPH1 in Mice (Studies AF-95, AF-100, AF-106, AF-110, AF-113, AF-117)

MNPH1 is highly attenuated in BALB/c mice. We have performed five experiments (AF-95, AF-100, AF-106, AF-110, AF-113) specifically to examine dissemination and persistence of bacteria after oral inoculation. Data from study AF-110 is found in Table ______. The bacterial inoculum used for these experiments was made from secondary research stocks. Bacteria were grown for 3 days on Brucella agar plates, scraped off, washed in 0.9% NaCl (saline), and adjusted in saline based on OD600 to an appropriate concentration for oral administration. Using a feeding needle, mice were given 0.2 ml of 2.5% sodium bicarbonate; 10-15 minutes later they were given 0.2 ml of MNPH1. Organs were harvested at various times from 1 day up to 12 weeks after inoculation to determine the extent of infection and eventual clearance of the vaccine from infected organs. The first two experiments used female mice; the last three examined male mice, since male genitalia are a target of Brucella infection. There were no apparent differences in infection of nongenital organs between male and female mice. When mice were given 10¹¹ CFU of MNPH1 orally by gavage, a transient bacteremia ensued.

In experiment AF-117, bacteremia was documented 1 day after inoculation of bacteria in 3 of 5 mice, but was not recovered from blood of 20 additional mice sampled in experiments AF-106 and AF-117 from 7 to 29 days after inoculation.

Summarized infection data in the spleen, lung and liver from these experiments are shown in the Tables 1, 2, and 3. As early as Day 1, bacteria were found in liver, lung, and spleen and continued to be isolated from these organs up to Day 31.

Bacteria were also recovered from cervical lymph nodes from Day 3 and persisted for up to 29 days after inoculation. These positive cervical lymph node cultures probably represent infection from pharyngeal or upper esophageal trauma or transmucosal infection. In addition, 1 of 5 mice in experiment AF-117 had bacteria recovered from epididymis at Day 1. No organisms were recovered from inguinal lymph nodes of this animal or from any of 59 additional mice examined up to 31 days after inoculation. The positive culture in epididymis from this mouse probably is a consequence of the concomitant Day 1 bacteremia. Except for 1 mouse at Day 53 in experiment AF-117, spleens, livers, lungs, and inguinal and cervical lymph nodes had no Brucella when sampled at Days 53-85.

Mouse #22 in experiment AF-117 had disseminated infection in lungs, liver, spleen, and inguinal and cervical lymph nodes, testis, and epididymis at Day 53. Spleen infection was accompanied by splenomegaly. The organism was presumptively identified as MNPH1 based on characteristic Brucella morphology, positive slide agglutination with Brucella antiserum, and kanamycin resistance, but unfortunately the culture died before further evaluation could be completed. No necropsy data other than spleen weight are available from this animal, so whether it represents a normal mouse is unknown. In contrast to MNPH1, wild-type parent strain B. melitensis 16M caused persistent disseminated infection of all organs tested through 85 days, with histopathological abnormalities in testicular vessels in 2 animals. These data indicated that MNPH1 is highly attenuated for infection of BALB/c mice.

TABLE 1
Summary Data: MNPH1 in Mouse Spleen

| | | | Organs | | | | |
|---|---|---|---|---|---|---|---|
| Days Post- | Log CFU | | No. | No. Non- | % | Infected Organs Log CFU | |
| Inoculation | Mean | Std. Dev. | Infected | Infected | Infected | Mean | Std. Dev. |
| 1-8 | 1.81 | 0.95 | 29 | 8 | 78 | 2.27 | 0.64 |
| 10-15 | 0.86 | 0.64 | 12 | 13 | 48 | 1.89 | 0.78 |
| 20-31 | 0.43 | 0.30 | 7 | 12 | 37 | 1.03 | 0.20 |
| 53-59 | 0.33 | 0.25 | 1 | 14 | 7 | 2.19 | n/a |
| 81-85 | 0.00 | 0.00 | 0 | 16 | 0 | n/a | n/a |

TABLE 2
Summary Data: MNPH1 in Mouse Liver

| | | | Organs | | | | |
|---|---|---|---|---|---|---|---|
| Days Post- | Log CFU | | No. | No. Non- | % | Infected Organs Log CFU | |
| Inoculation | Mean | Std. Dev. | Infected | Infected | Infected | Mean | Std. Dev. |
| 1-8 | 0.99 | 0.61 | 19 | 16 | 54 | 1.79 | 0.45 |
| 10-15 | 0.24 | 0.24 | 3 | 20 | 13 | 1.79 | 0.39 |
| 20-31 | 0.37 | 0.27 | 2 | 16 | 11 | 1.52 | 1.05 |
| 53-59 | 0.22 | 0.17 | 1 | 14 | 7 | 1.48 | n/a |
| 81-85 | 0.00 | 0.00 | 0 | 16 | 0 | n/a | n/a |

TABLE 3
Summary Data: MNPH1 in Mouse Lung

| | | | Organs | | | | |
|---|---|---|---|---|---|---|---|
| Days Post- | Log CFU | | No. | No. Non- | % | Infected Organs Log CFU | |
| Inoculation | Mean | Std. Dev. | Infected | Infected | Infected | Mean | Std. Dev. |
| 1-8 | 1.17 | 0.67 | 21 | 15 | 58 | 2.11 | 0.95 |
| 10-15 | 0.92 | 0.66 | 11 | 12 | 48 | 1.83 | 1.00 |
| 20-31 | 0.34 | 0.28 | 4 | 15 | 21 | 0.81 | 0.36 |
| 53-59 | 0.27 | 0.21 | 1 | 14 | 7 | 1.78 | n/a |
| 81-85 | 0.00 | 0.00 | 0 | 16 | 0 | n/a | n/a |

TABLE 4
Mouse Study AF-110 Data Tables

| | | | | | |
|---|---|---|---|---|---|
| Days | | | | No. | |
| after | Exper. | Mean Log | No. | Non- | % |

| | | | | | | |
|---|---|---|---|---|---|---|
| Immun. | Group | CFU | SD | Infect. | Infect. | Infect. |

Table 4A - Lung Data

| | | | | | | |
|---|---|---|---|---|---|---|
| 3 | 16M | 2.60 | 2.39 | 3 | 2 | 60 |
| | MNPH1 | 0.54 | 1.21 | 1 | 4 | 20 |
| 7 | 16M | 3.40 | 0.22 | 5 | 0 | 100 |
| | MNPH1 | 1.59 | 2.44 | 2 | 3 | 40 |
| 14 | 16M | 3.51 | 1.36 | 5 | 0 | 100 |
| | MNPH1 | 1.60 | 1.13 | 3 | 1 | 75 |
| 31 | 16M | 1.73 | 1.03 | 4 | 1 | 80 |
| | MNPH1 | 0.12 | 0.27 | 1 | 4 | 20 |
| 57 | 16M | 2.73 | 0.90 | 5 | 0 | 100 |
| | MNPH1 | 0.00 | 0.00 | 0 | 5 | 0 |
| 85 | 16M | 1.19 | 1.69 | 2 | 3 | 40 |
| | MNPH1 | 0.00 | 0.00 | 0 | 5 | 0 |

Table 4B - Liver Data

| | | | | | | |
|---|---|---|---|---|---|---|
| 3 | 16M | 2.24 | 1.92 | 4 | 1 | 80 |
| | MNPH1 | 0.73 | 1.27 | 1 | 2 | 33 |
| 7 | 16M | 3.58 | 0.41 | 5 | 0 | 100 |
| | MNPH1 | 0.85 | 1.18 | 2 | 3 | 40 |
| 14 | 16M | 2.95 | 0.61 | 5 | 0 | 100 |
| | MNPH1 | 0.00 | 0.00 | 0 | 4 | 0 |
| 31 | 16M | 2.07 | 1.26 | 4 | 1 | 80 |
| | MNPH1 | 0.00 | 0.00 | 0 | 4 | 0 |
| 57 | 16M | 2.20 | 1.32 | 4 | 1 | 80 |
| | MNPH1 | 0.00 | 0.00 | 0 | 5 | 0 |
| 85 | 16M | 1.37 | 1.39 | 3 | 2 | 60 |
| | MNPH1 | 0.00 | 0.00 | 0 | 5 | 0 |

| | | | | | | Organs | | |
|---|---|---|---|---|---|---|---|---|
| Days | | | | | | # | # | |
| after | Exper. | Spleen Weight | | Log CFU | | In- | Non- | % |
| Immun. | Group | Mean | SD | Mean | SD | fect | Infect. | Infect. |

Table 4C - Spleen Data

| | | | | | | | | |
|---|---|---|---|---|---|---|---|---|
| 3 | 16M | 0.11 | 0.03 | 2.75 | 0.69 | 5 | 0 | 100 |
| | MNPH1 | 0.10 | 0.01 | 1.16 | 1.08 | 4 | 0 | 100 |
| 7 | 16M | 0.12 | 0.02 | 4.22 | 0.23 | 5 | 0 | 100 |
| | MNPH1 | 0.10 | 0.02 | 1.66 | 0.97 | 4 | 1 | 80 |
| 14 | 16M | 0.25 | 0.09 | 4.88 | 0.69 | 5 | 0 | 100 |
| | MNPH1 | 0.09 | 0.02 | 0.54 | 1.21 | 1 | 4 | 20 |
| 31 | 16M | 0.26 | 0.03 | 3.95 | 0.09 | 5 | 0 | 100 |
| | MNPH1 | 0.09 | 0.01 | 0.00 | 0.00 | 0 | 5 | 0 |
| 57 | 16M | 0.33 | 0.09 | 4.69 | 0.76 | 5 | 0 | 100 |
| | MNPH1 | 0.08 | 0.02 | 0.00 | 0.00 | 0 | 5 | 0 |
| 85 | 16M | 0.25 | 0.16 | 3.66 | 0.90 | 4 | 0 | 100 |
| | MNPH1 | 0.11 | 0.03 | 0.00 | 0.00 | 0 | 5 | 0 |

| | | | | | |
|---|---|---|---|---|---|
| Days | | | | No. | |
| after | Exper. | Log CFU | No. | Non- | % |

| | | | | | | |
|---|---|---|---|---|---|---|
| Immun. | Group | Mean | SD | Infect. | Infect. | Infect. |

Table 4D - Cervical Lymph Node Data

| | | | | | | |
|---|---|---|---|---|---|---|
| 3 | 16M | 1.52 | 1.71 | 3 | 2 | 60 |
| | MNPH1 | 0.15 | 0.30 | 2 | 3 | 40 |
| 7 | 16M | 3.81 | 0.25 | 5 | 0 | 100 |
| | MNPH1 | 1.12 | 1.28 | 3 | 2 | 60 |
| 14 | 16M | 3.95 | 0.16 | 5 | 0 | 100 |
| | MNPH1 | 2.07 | 1.41 | 4 | 1 | 80 |
| 31 | 16M | 0.49 | 1.09 | 1 | 4 | 20 |
| | MNPH1 | 0.00 | 0.00 | 0 | 5 | 0 |
| 57 | 16M | 0.31 | 0.70 | 1 | 4 | 20 |
| | MNPH1 | 0.00 | 0.00 | 0 | 5 | 0 |
| 85 | 16M | 2.60 | 0.50 | 5 | 0 | 100 |
| | MNPH1 | 0.00 | 0.00 | 0 | 5 | 0 |

Table 4E - Inguinal Lymph Node Data

| | | | | | | |
|---|---|---|---|---|---|---|
| 3 | 16M | 0.06 | 0.13 | 1 | 4 | 20 |
| | MNPH1 | 0.00 | 0.00 | 0 | 5 | 0 |
| 7 | 16M | 0.34 | 0.61 | 2 | 3 | 40 |
| | MNPH1 | 0.00 | 0.00 | 0 | 5 | 0 |
| 14 | 16M | 1.90 | 2.01 | 3 | 2 | 60 |
| | MNPH1 | 0.00 | 0.00 | 0 | 5 | 0 |
| 31 | 16M | 1.01 | 0.66 | 4 | 1 | 80 |
| | MNPH1 | 0.00 | 0.00 | 0 | 5 | 0 |
| 57 | 16M | 3.04 | 2.23 | 4 | 1 | 80 |
| | MNPH1 | 0.00 | 0.00 | 0 | 5 | 0 |
| 85 | 16M | 1.81 | 1.13 | 4 | 1 | 80 |
| | MNPH1 | 0.00 | 0.00 | 0 | 5 | 0 |

| Days | | | Organs | | |
|---|---|---|---|---|---|
| after | Exper. | Log CFU | | # Non- | % |

| | | | | | | |
|---|---|---|---|---|---|---|
| Immun. | Group | Mean | SD | No. Infect | Infect. | Infect. |

Table 4F - Epididymis Data

| | | | | | | |
|---|---|---|---|---|---|---|
| 3 | 16M | 0.00 | 0.00 | 0 | 4 | 0 |
| | MNPH1 | 0.00 | 0.00 | 0 | 5 | 0 |
| 7 | 16M | 0.20 | 0.45 | 1 | 4 | 20 |
| | MNPH1 | 0.00 | 0.00 | 0 | 5 | 0 |
| 14 | 16M | 0.78 | 1.23 | 2 | 3 | 40 |
| | MNPH1 | 0.00 | 0.00 | 0 | 5 | 0 |
| 31 | 16M | 0.52 | 1.16 | 1 | 4 | 20 |
| | MNPH1 | 0.00 | 0.00 | 0 | 5 | 0 |
| 57 | 16M | 0.30 | 0.67 | 1 | 4 | 20 |
| | MNPH1 | 0.00 | 0.00 | 0 | 5 | 0 |
| 85 | 16M | 1.89 | 0.66 | 4 | 0 | 100 |
| | MNPH1 | 0.00 | 0.00 | 0 | 5 | 0 |

Table 4G - Testis Data

| | | | | | | |
|---|---|---|---|---|---|---|
| 3 | 16M | 0.00 | 0.00 | 0 | 5 | 0 |
| | MNPH1 | 0.00 | 0.00 | 0 | 5 | 0 |
| 7 | 16M | 0.38 | 0.85 | 1 | 4 | 20 |
| | MNPH1 | 0.00 | 0.00 | 0 | 5 | 0 |
| 14 | 16M | 0.68 | 0.82 | 3 | 2 | 60 |
| | MNPH1 | 0.00 | 0.00 | 0 | 5 | 0 |
| 31 | 16M | 0.06 | 0.13 | 1 | 4 | 20 |
| | MNPH1 | 0.00 | 0.00 | 0 | 5 | 0 |
| 57 | 16M | 0.29 | 0.57 | 1 | 3 | 25 |
| | MNPH1 | 0.00 | 0.00 | 0 | 5 | 0 |
| 85 | 16M | 2.13 | 1.60 | 3 | 1 | 75 |
| | MNPH1 | 0.00 | 0.00 | 0 | 5 | 0 |

Fecal Excretion of Vaccine by Mice (Studies AF-106, AF-117)

In two experiments, we determined fecal excretion of MNPH1 beginning on Day 1 after inoculation. In experiment AF-106, feces were positive on Day 1 and were not sampled again until Days 8 through 11, when they were negative. In experiment AF-117, feces were positive through Day 9, but fecal cultures were negative for Brucella at Days 10 and 11 (Table 5).

TABLE 5
Fecal Excretion of MNPH1 after Oral Inoculation (AF-117)

| Days Post- | |
|---|---|
| Inoculation | # Positive/Total |
| 1 | 5/5 |
| 2 | 4/5 |
| 3 | 3/5 |
| 4 | 3/5 |
| 8 | 2/5 |
| 9 | 2/5 |
| 10 | 0/5 |
| 11 | 0/5 |

Immunogenicity and Efficacy in Mice (Studies AF-103, AF-106, AF-100, AF-112, AF-113, AF-117)

Oral inoculation of mice one time with MNPH1 led to production of anti-Brucella LPS IgG (AF-100, AF-106, AF-117). Results from experiment AF-100 are summarized in Table 6.

TABLE 6
Anti-B. melitensis IgG titers (AF-100)

| Day Post- | Anti-LPS IgG, |
|---|---|
| Inoculation | Mean ± SD OD units |
| 3 | 1.6 ± .89 |
| 7 | 86 ± 62 |
| 10 | 104 ± 59 |
| 14 | 642 ± 897 |
| 20 | 1114 ± 852 |
| 25 | 1077 ± 745 |

They also produced IL-2 (AF-103, AF-100) and IFN-γ (AF-100) in supernatants of antigen-stimulated spleen mononuclear cell cultures. In experiment AF-100, MNPH1-inoculated mice secreted 45±31 pg/mL of IL-2 vs 0±0 (<15) pg/ml for sham (saline)-inoculated mice (p<0.0001). Similarly, MNPH1-inoculated mice secreted 1368±793 pg/ml of IFN-γ vs 610±672 pg/ml for sham (saline)-inoculated mice (p<0.092). Spleen cells cultured with medium alone secreted little or no IL-2 and IFN-γ.

Forty percent of mice inoculated once with 10¹¹ CFU of MNPH1 were protected against dissemination to spleen when challenged intranasally with 16M (AF-103). Seventy percent of mice inoculated three times were protected (AF-103). Similar trends were seen for prevention of dissemination to the liver. Although reduced clearance of bacteria from the lung was observed at 2 weeks in mice inoculated once or three times, the effect persisted at 8 weeks only in mice inoculated once.

Inoculation with two or three doses also led to production of anti-LPS IgG and antigen-stimulated spleen cell production of IL-2 and IFN-γ (results not presented). Antigen-stimulated cytokine production persisted in spleen cells harvested 24 weeks after the last dose of a three-dose vaccination schedule. Inoculation with three doses led to 60%-70% protection from dissemination of organisms to the spleen at 8 weeks. Inoculation with two doses led to 46% protection in a single experiment. A trend toward protection for dissemination to the spleen persisted up to 24 weeks after the third dose of MNPH1. Vaccination showed similar trends toward protection from dissemination to the liver. In two of three experiments, vaccination also enhanced clearance of bacteria from the lungs.

The above data indicated that oral inoculation of MNPH1 leads to anti-Brucella humoral and cellular immune responses, protection from disseminated infection, and a trend toward enhanced clearance of challenge bacteria from lungs. Select summary data tables from studies AF-103, AF-112, and AF-113 are presented in Tables 7, 8, 9 and 10.

TABLE 7
Mouse Study AF-103 Data Tables

| | | | | | No. | No. | |
|---|---|---|---|---|---|---|---|
| Days after | | Exper. | Mean Log | | In- | Non- | % |
| Immun. | Chall. | Group | CFU | SD | fect. | Infect. | Infect. |

Table 7A - Lung Data

| | | | | | | | |
|---|---|---|---|---|---|---|---|
| 51 | −1 | MNPH1(1×) | | | | | |
| | | MNPH1(3×) | | | | | |
| 53 | 1 | MNPH1(1×) | 2.00 | 0.95 | 5 | 0 | 100 |
| | | MNPH1(3×) | 2.61 | 0.21 | 5 | 0 | 100 |
| | | Saline | 2.83 | 0.29 | 5 | 0 | 100 |
| 59 | 7 | MNPH1(1×) | 2.34 | 1.11 | 4 | 0 | 100 |
| | | MNPH1(3×) | 2.39 | 0.60 | 5 | 0 | 100 |
| | | Saline | 2.57 | 0.56 | 4 | 0 | 100 |
| 66 | 14 | MNPH1(1×) | 1.67 | 0.94 | 4 | 1 | 80 |
| | | MNPH1(3×) | 1.73 | 0.52 | 3 | 0 | 100 |
| | | Saline | 3.02 | 0.61 | 4 | 0 | 100 |
| 108 | 56 | MNPH1(1×) | 0.62 | 0.75 | 8 | 8 | 50 |
| | | MNPH1(3×) | 1.10 | 1.33 | 7 | 8 | 47 |
| | | Saline | 1.30 | 1.03 | 10 | 5 | 67 |

Table 7B - Liver Data

| 51 | −1 | MNPH1(1×) | | | | | |
|---|---|---|---|---|---|---|---|
| | | MNPH1(3×) | | | | | |
| 53 | 1 | MNPH1(1×) | 0.00 | 0.00 | 0 | 5 | 0 |
| | | MNPH1(3×) | 0.00 | 0.00 | 0 | 5 | 0 |
| | | Saline | 0.00 | 0.00 | 0 | 5 | 0 |
| 59 | 7 | MNPH1(1×) | 0.00 | 0.00 | 0 | 5 | 0 |
| | | MNPH1(3×) | 0.31 | 0.54 | 2 | 3 | 40 |
| | | Saline | 0.00 | 0.00 | 5 | 0 | 100 |
| 66 | 14 | MNPH1(1×) | 0.92 | 0.84 | 3 | 2 | 60 |
| | | MNPH1(3×) | 0.53 | 0.91 | 1 | 2 | 33 |
| | | Saline | 1.44 | 1.15 | 4 | 0 | 100 |
| 108 | 56 | MNPH1(1×) | 1.17 | 1.05 | 10 | 6 | 63 |
| | | MNPH1(3×) | 0.43 | 0.89 | 4 | 11 | 27 |
| | | Saline | 1.35 | 1.27 | 10 | 5 | 67 |
| | | | | | No. | No. | |
| Days after | | Exper. | Log CFU | | In- | Non- | % |
| Immun. | Chall. | Group | Mean | SD | fect. | Infect. | Infect. |

Table 7C - Spleen Data

| | | | | | | | |
|---|---|---|---|---|---|---|---|
| 51 | −1 | MNPH1(1×) | 0.00 | 0.00 | 0 | 3 | 0 |
| | | MNPH1(3×) | 0.00 | 0.00 | 0 | 3 | 0 |
| 53 | 1 | MNPH1(1×) | 0.00 | 0.00 | 0 | 5 | 0 |
| | | MNPH1(3×) | 0.00 | 0.00 | 0 | 5 | 0 |
| | | Saline | 0.00 | 0.00 | 0 | 5 | 0 |
| 59 | 7 | MNPH1(1×) | 0.00 | 0.00 | 0 | 5 | 0 |
| | | MNPH1(3×) | 0.68 | 0.97 | 2 | 3 | 40 |
| | | Saline | 0.22 | 0.34 | 2 | 3 | 40 |
| 66 | 14 | MNPH1(1×) | 1.27 | 0.90 | 4 | 1 | 80 |
| | | MNPH1(3×) | 0.72 | 1.24 | 1 | 2 | 33 |
| | | Saline | 1.53 | 1.13 | 3 | 1 | 75 |
| 108 | 56 | MNPH1(1×) | 1.42 | 1.34 | 9 | 7 | 56 |
| | | MNPH1(3×) | 0.84 | 1.54 | 4 | 11 | 27 |
| | | Saline | 3.46 | 1.01 | 15 | 0 | 100 |

TABLE 8
Production of IFN-gamma, Study AF-112
Spleen Data

| | | |
|---|---|---|
| | Experimental | |
| Days after | Condition | IFN-g, pg/ml |

| Immun. | Chall. | Description | No. | Mean | SD |
|---|---|---|---|---|---|
| 57 | −1 | | | | |
| Con A | | | | | |
| | | Saline | 1 | | |
| | | MNPH1 2× | 2 | | |
| | | MNPH1 3× | 3 | | |
| Medium | | | | | |
| | | Saline | 1 | 0 | 0 |
| | | MNPH1 2× | 2 | 0 | 0 |
| | | MNPH1 3× | 3 | 0 | 0 |
| RFBU | | | | | |
| | | Saline | 1 | 1732 | 630 |
| | | MNPH1 2× | 2 | 15375 | 8004 |
| | | MNPH1 3× | 3 | 6489 | 6842 |

ConA = concanavalin A. This is a nonspecific stimulator of T cell responses.
RFBL = a whole bacterial lysate of rough Brucella strain WRR51

TABLE 9
Production of IL-2, Study AF-112 Spleen Data

| | | |
|---|---|---|
| | Experimental | |
| Days after | Condition | IFN-g, pg/ml |

| Immun. | Chall. | Description | No. | Mean | SD |
|---|---|---|---|---|---|
| 57 | −1 | | | | |
| Con A | | | | | |
| | | Saline | 1 | 1430 | 460 |
| | | MNPH1 2× | 2 | 1357 | 276 |
| | | MNPH1 3× | 3 | 1513 | 458 |
| Medium | | | | | |
| | | Saline | 1 | 0 | 0 |
| | | MNPH1 2× | 2 | 0 | 0 |
| | | MNPH1 3× | 3 | 0 | 0 |
| RFBU | | | | | |
| | | Saline | 1 | 22 | 48 |
| | | MNPH1 2× | 2 | 250 | 71 |
| | | MNPH1 3× | 3 | 306 | 131 |

ConA = concanavalm A. This is a nonspecific stimulator of T cell responses.
RFBL = a whole bacterial lysate of rough Brucella strain WRR51

TABLE 10
Mouse Study AF-113 Data Tables
Spleen Data

| | | | | | Organs | | |
|---|---|---|---|---|---|---|---|
| Days after | | Exper. | Log CFU | | # | # Non- | % |
| Immun. | Chall. | Group | Mean | SD | Infect. | Infect. | Infect. |
| −168 | 1 | MNPH1 | 0.06 | 0.13 | 1 | 4 | 20 |
| | | Saline | 0.00 | 0.00 | 0 | 5 | 0 |
| | 28 | MNPH1 | 0.51 | 0.89 | 3 | 7 | 30 |
| | | Saline | 2.39 | 0.62 | 10 | 0 | 100 |
| | 56 | MNPH1 | 2.44 | 1.65 | 12 | 3 | 80 |
| | | Saline | 3.52 | 1.48 | 13 | 1 | 93 |
| −112 | 1 | MNPH1 | 0.00 | 0.00 | 0 | 5 | 0 |
| | | Saline | 0.00 | 0.00 | 0 | 5 | 0 |
| | 28 | MNPH1 | 0.48 | 0.76 | 4 | 6 | 40 |
| | | Saline | 2.25 | 1.00 | 9 | 0 | 100 |
| | 56 | MNPH1 | 2.04 | 1.89 | 6 | 2 | 75 |
| | | Saline | 4.25 | 0.92 | 14 | 0 | 100 |
| −84 | 1 | MNPH1 | 0.06 | 0.13 | 1 | 4 | 20 |
| | | Saline | 0.00 | 0.00 | 0 | 5 | 0 |
| | 28 | MNPH1 | 0.38 | 0.78 | 2 | 7 | 22 |
| | | Saline | 1.96 | 1.13 | 9 | 1 | 90 |
| | 56 | MNPH1 | 1.01 | 1.70 | 7 | 8 | 47 |
| | | Saline | 2.88 | 2.38 | 10 | 5 | 67 |
| −56 | 1 | MNPH1 | 0.00 | 0.00 | 0 | 5 | 0 |
| | | Saline | 0.00 | 0.00 | 0 | 5 | 0 |
| | 28 | MNPH1 | 0.20 | 0.39 | 3 | 7 | 30 |
| | | Saline | 2.86 | 0.93 | 5 | 0 | 100 |
| | 56 | MNPH1 | 1.10 | 1.80 | 3 | 5 | 38 |
| | | Saline | 4.20 | 1.04 | 14 | 0 | 100 |

Negative values denote days prior to challenge.

### Example 3

### Non-Human Primate Studies

Safety Studies in Rhesus Macaques (Studies WR9901, IO05-02, IO10-O₂)

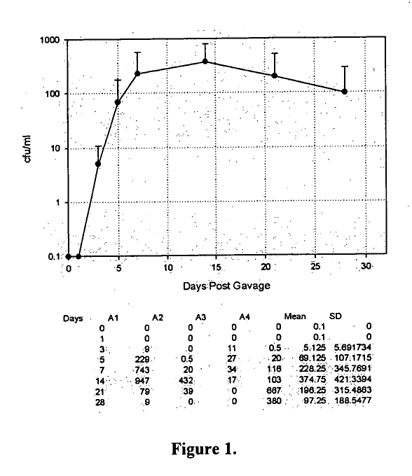

In experiment WR9901 (FIG. 1), the course of disease due to virulent B. melitensis 16M, the parent strain of MNPH1, was studied in four Rhesus macaques for 28 days after administration of an actual dose of 1.0×10¹² CFU orally by gavage. Bacteria were prepared by scraping, washing, and resuspension from a 3-day culture on Brucella agar plates. Animals were followed clinically and serial twice-weekly blood cultures and analysis of a panel of 14 serum proteins was performed. Animals were necropsied 28 days after administration of 16M. Animals appeared severely ill by the end of the study. Three of four animals became bacteremic by Day 3 and all were bacteremic by Day 5.

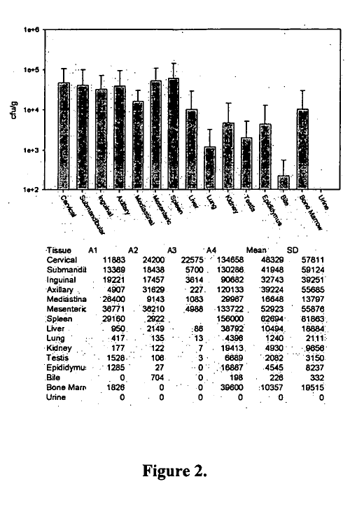

Bacteremia persisted in all animals through Day 21, two of four monkeys were still bacteremic at the termination of the study on Day 28 (FIG. 2). Peak levels of bacteremia for the four animals ranged from 34 to 947 CFU/ml of blood. At necropsy, mean recovery of 16M from all lymph node groups sampled was at least 10⁴ CFU per gram. Spleen, liver, and bone marrow had similarly high numbers of organisms. In addition, 16M was recovered from lung, liver, kidney, testis, and epididymis. One animal (A3) was found at necropsy to be asplenic due to a splenectomy that was not recognized prior to inclusion in the study. Interestingly, this animal had markedly less severe bacteremia and consistently lower numbers of organisms recovered from all tissues except cervical lymph nodes.

A panel of 14 serum assays (glucose, BUN, creatinine, total protein, albumin, calcium, phosphorus, total bilirubin, AST (aspartate aminotransferase), ALT (alanine aminotransferase), LDH (lactic dehydrogenase), alkaline phophatase, gamma glutamyl transferase (GGT), and CPK (creatine phosphokinase)) done at each time point were generally unrevealing, except that three animals had at least twofold elevations of alkaline phosphatase and one had a twofold increase in GGT on either Day 21 or Day 28.

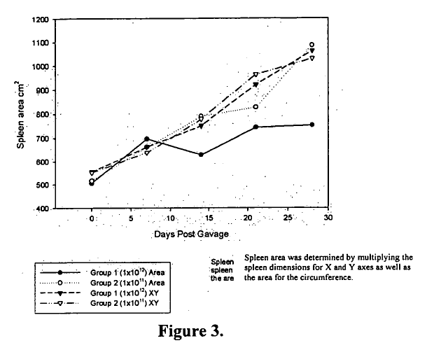

In study IO05-02 (FIG. 3), two groups of four Rhesus macaques each were given 10¹¹ (actual dose 9.6×10¹⁰) or 10¹² (actual dose 9.4×10¹¹) CFU of MNPH1 orally by gavage with sodium bicarbonate. Bacteria were prepared by scraping, washing, and resuspension from a 3-day culture on Brucella agar plates. Temperature was recorded for 21 days using an implanted recording device. Spleens were palpated and cross-sectional area estimated by ultrasonography. Feces were cultured daily for the first week and twice weekly thereafter. Blood was obtained twice weekly and all animals were necropsied for pathological examination 28 days after inoculation.

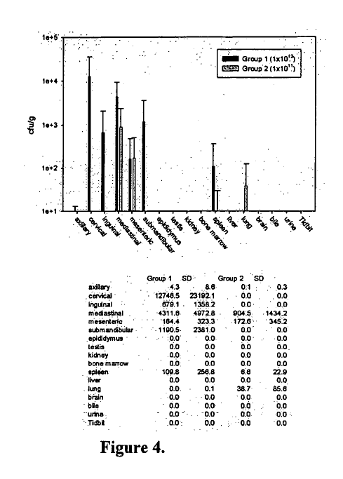

Animals in both groups appeared healthy throughout the study. Animals in both groups developed increased spleen size by palpation and ultrasonography. Axillary and inguinal, but not cervical, lymph nodes became enlarged (generally 1 to 2+ on a scale of 4) in all animals (FIG. 4). Temperature data were difficult to interpret due to artifacts, but suggested increased temperatures at the time of bacteremia, especially in the high-dose group. Animals in both groups lost up to 9% of their body weight during the study, attributable in part to reduced feeding. All four animals in the 10¹² group were bacteremic at Day 7 only. One animal in the 10¹¹ group was bacteremic at Day 4 only and another at Day 7 only. Bacterial counts in blood were 0.5-1 CFU/ml. At necropsy, bacteria were recovered from one to three lymph nodes of all animals in the high-dose group and from one lymph node each from three of four animals in the low-dose group. In the high-dose group, spleens were also infected in two animals, but no other organs were infected. In the low-dose group, one animal had no Brucella recovered from nodes or other organs. One animal was infected in nodes and spleen only; another was infected in nodes and lung only. The intensity of infection of these organs was less in the animals in the low-dose group.

No organisms were recovered from liver, testes, brain, kidney, bone marrow, or bile. Animals had a number of nonspecific histopathologic abnormalities, but none attributable to brucellosis except splenic lymph node hyperplasia in two of four animals in the low-dose group. The panel of 14 serum assays was unremarkable throughout the study. Brucella was shed in the feces of the high-dose group for 3 days and in the low-dose group for 2 days. In comparison with animals in the 16M study described above, these animals showed markedly less clinical and bacteriologic evidence of brucellosis.

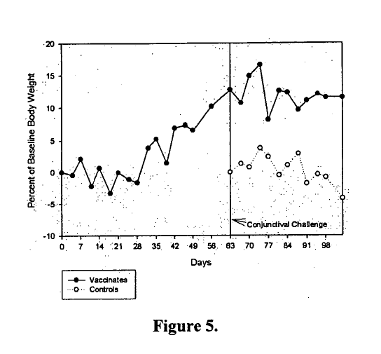

In experiment IO10-02 (FIG. 5), eight Rhesus macaques were given a target dose of 1×10¹¹ CFU (actual delivered dose 2×10¹¹ CFU) MNPH1 orally in bicarbonate. Bacteria were prepared by scraping, washing, and resuspension from a 3-day culture on Brucella agar plates. Animals appeared clinically well and remained afebrile after inoculation. In contrast to 1005-O₂, animals gained weight during this study.

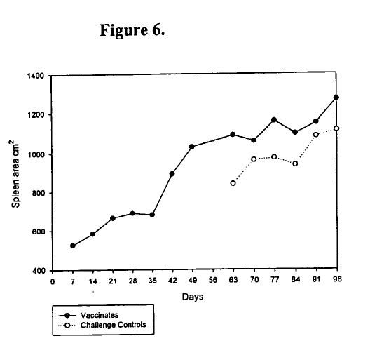

As noted in 1005-O₂, enlargement of axillary and inguinal nodes developed after immunization (FIG. 6), with severity of lymphadenopathy assessed as 1+ to 2+on a scale of 4. Splenic enlargement also occurred.

Seven of eight animals became bacteremic. Six of these animals had positive blood cultures for 4 days or less (two consecutive sample times). Three were only positive on one occasion. Animal A2 had positive blood cultures at four consecutive sample times from Days 4 to 18. No MNPH1 was recovered from any animal's blood after 18 days. Of the 14 positive blood samples, only 3 grew directly on Brucella agar plates, with low colony counts (2, 2, and 3 CFU/ml). In the other 11 positive samples, bacteria were recovered only from broth cultures. Four animals were necropsied 8 weeks after immunization. Bacteria were recovered only from submandibular lymph nodes in animals A3 and A4, from submandibular and axillary lymph nodes and spleen from animal A2, and from submandibular, mediastinal, and mesenteric lymph nodes and lung from animal A4. In lymph nodes, MNPH1 concentrations ranged from 5 CFU/g to 4×10⁴ CFU/g, but were less than 20 CFU/g in the single isolations from spleen and lung. MNPH1 was not recovered from cervical or inguinal nodes or from liver, testes, brain, kidney, bone marrow, bile, urine or the implanted temperature-monitoring device.

Nine weeks after vaccination, the four remaining vaccinates were challenged conjunctivally with 16M, as described below. At necropsy 6 weeks after challenge with 16M, culture of organs at necropsy disclosed 16M, but only one animal had persistent MNPH1. In that animal, the vaccine strain was recovered from the broth culture of a mediastinal lymph node. These data indicate that MNPH 1 causes minimal bacteremia at an oral dose of 2×10¹¹ CFU, persists in low levels in tissues of the mononuclear phagocyte system for at least 8 weeks, but is essentially cleared from vaccinates by 15 weeks. There is no evidence of infection of organs outside the mononuclear phagocyte system. These data indicate that, at oral doses of 10¹¹ to 10¹² CFU, MNPH1, while causing disseminated infection of the mononuclear phagocyte system sufficient to induce serum anti-LPS IgG, causes mild lymphadenopathy and splenic enlargement, but no infection of organs outside the mononuclear phagocyte system. It is markedly attenuated relative to wild-type parent strain 16M. In addition, it appears attenuated relative to orally administered B. melitensis Rev1 in a study done by Chen et al. in Cynomolgus monkeys (Chen and Elberg, 1970, “Immunization against Brucella infections: immune response of mice, guinea pigs, and Cynomolgus phillipinensis to live and killed Brucella melitensis strain Rev.I administered by various methods.” J. Infect. Dis. 122:489-500.). In those studies, a dose of 3.3×10¹¹ CFU caused bacteremia in two of two monkeys. One was positive from Days 7 through 50. The other was positive on Days 10 and 14. In that same Chen et al. study, two of two monkeys given 3.3×10⁹ CFU were bacteremic, one on Days 17, 22, and 36 and one on Days 3, 7, and 10. While we do not know the relative susceptibility of Rhesus, Cynomolgus, and humans to infection with various strains of B. melitensis, the frequency and duration of bacteremia induced by MNPH1 in our studies appear to be less than that observed by Chen et al. In our studies, an apparent dose-response effect on intensity of colonization of lymph nodes and other organs was observed. In addition, the intensity of infection in animals inoculated with 10¹¹ CFU and euthanized at 8 weeks post-inoculation (IO10-O₂) was lower than that observed in animals euthanized at 4 weeks (IO05—O₂). These data suggest that reduced doses will have less likelihood to cause disease or persistent infection in humans.

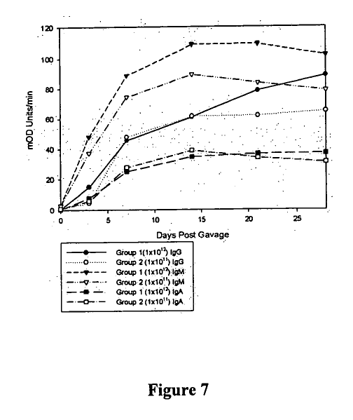

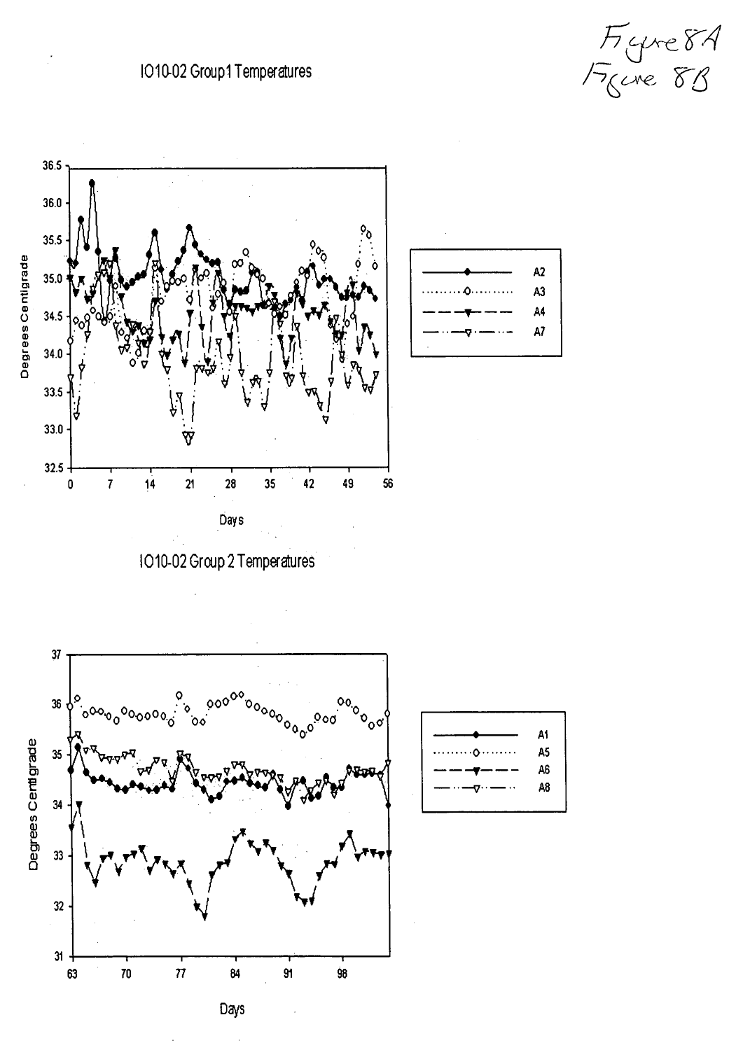

Immunogenicity and Efficacy Studies in Nonhuman Primates: (Studies IO05-02 and IO10-O₂). In experiment IO05-02 (FIG. 7), four Rhesus macaques given 10¹¹ CFU and four animals given 10¹² CFU of MNPH1 developed serum IgG, IgA, and IgM anti-LPS antibody. Similarly, all eight animals vaccinated in experiment IO10-02 developed antibody, which showed an anamnestic response after challenge.

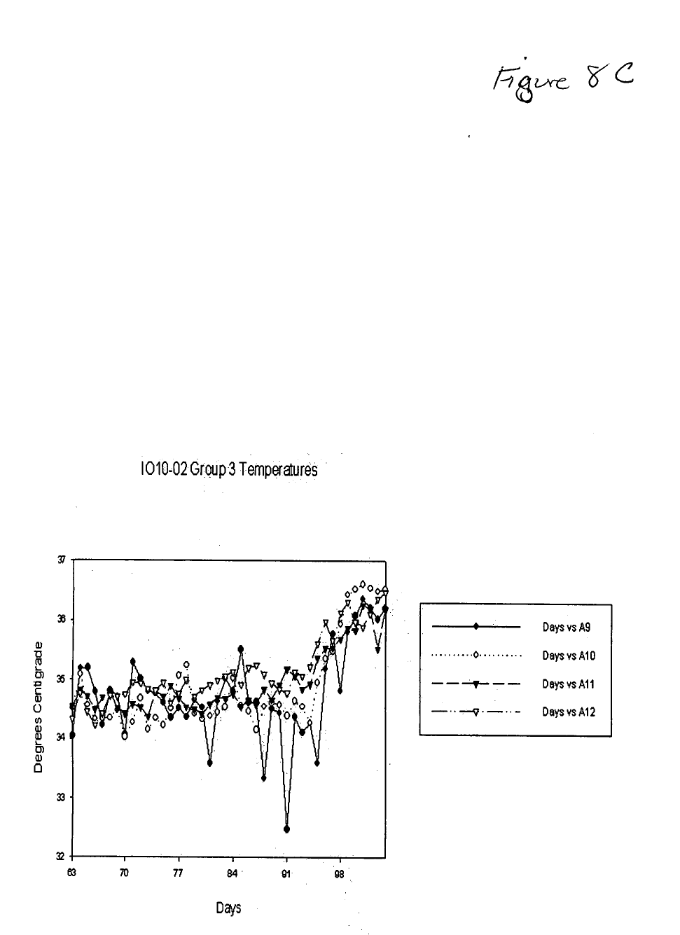

The four vaccinated animals that remained after necropsy in experiment IO10-02 and four additional, nonvaccinated Rhesus macaques were challenged 9 weeks after oral inoculation by conjunctival instillation of 10⁷ CFU of 16M. After challenge, all 4 nonvaccinated, but none of the vaccinated animals, became febrile (FIG. 8) (P=0.014, Fisher's Exact test).

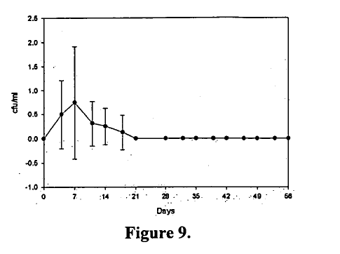

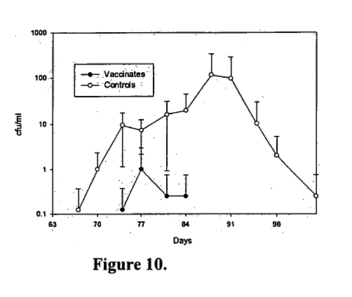

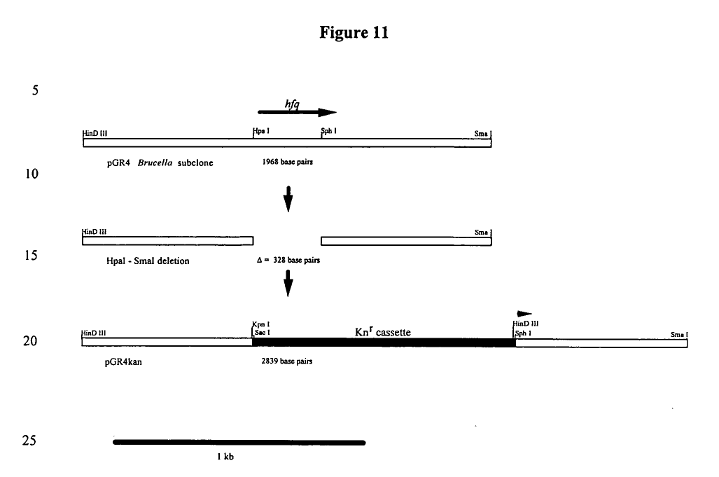

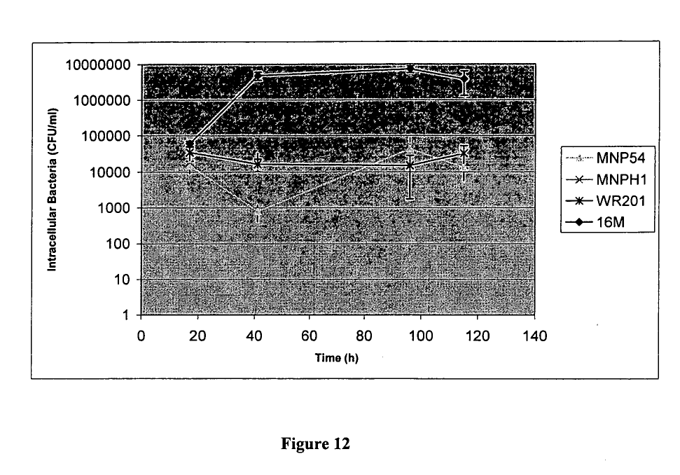

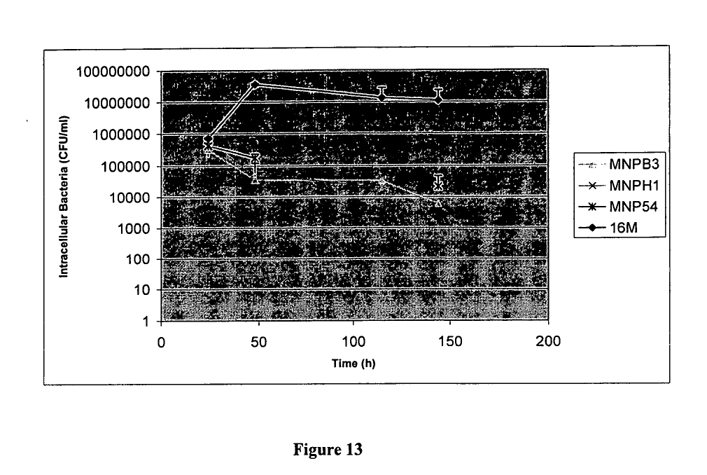

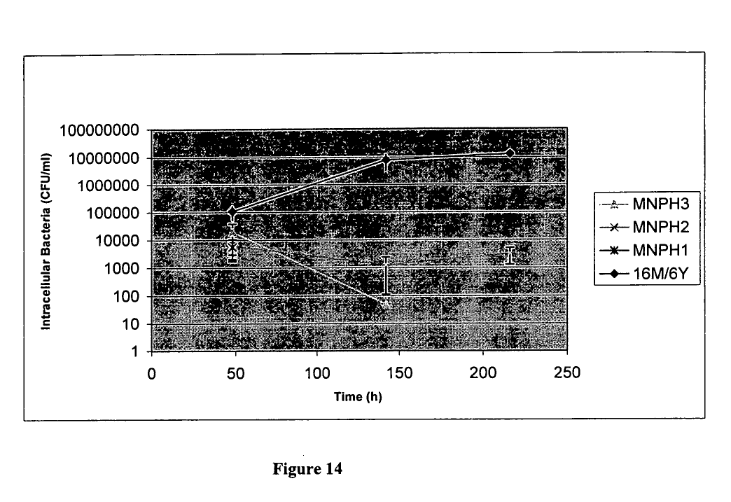

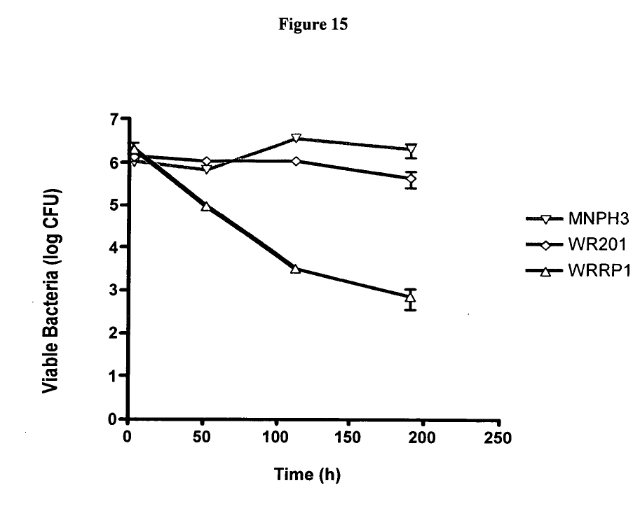

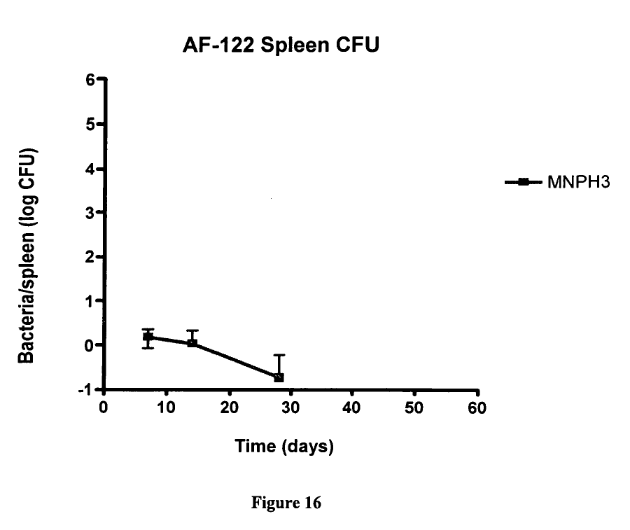

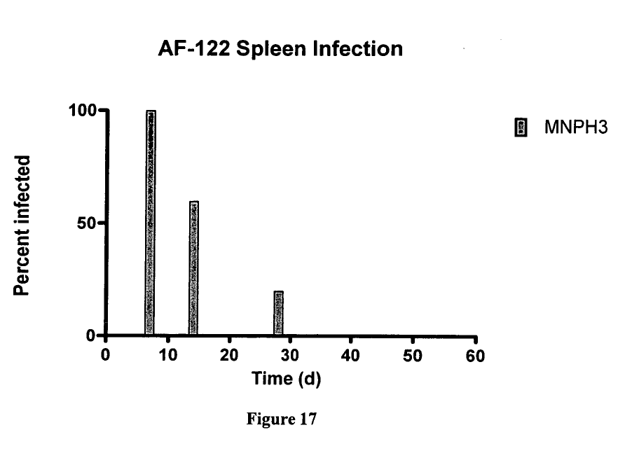

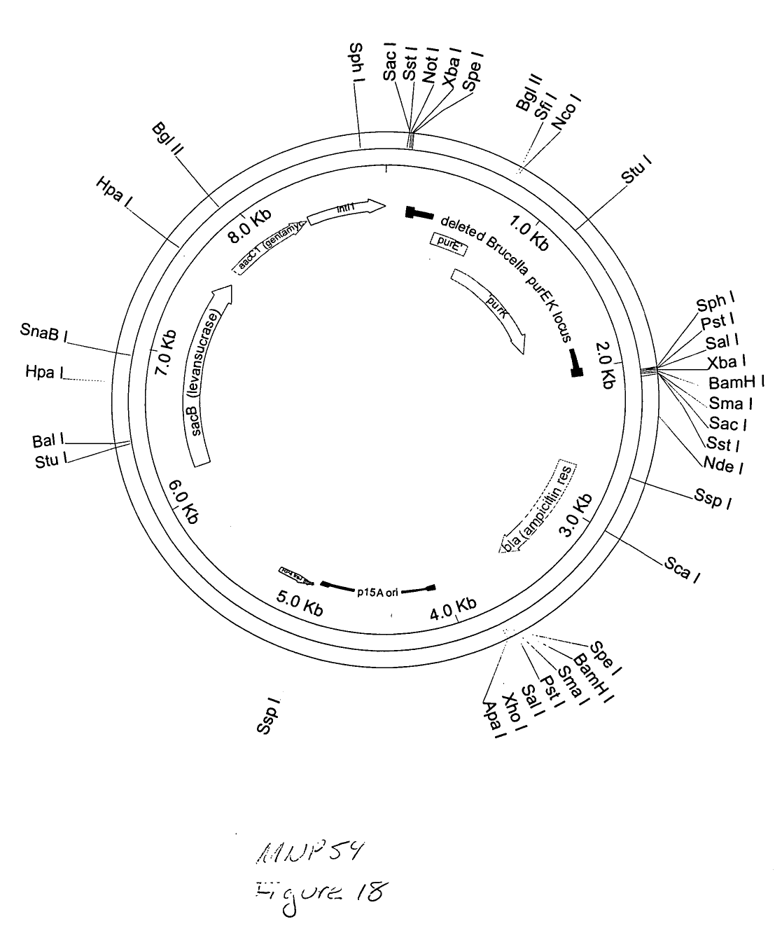

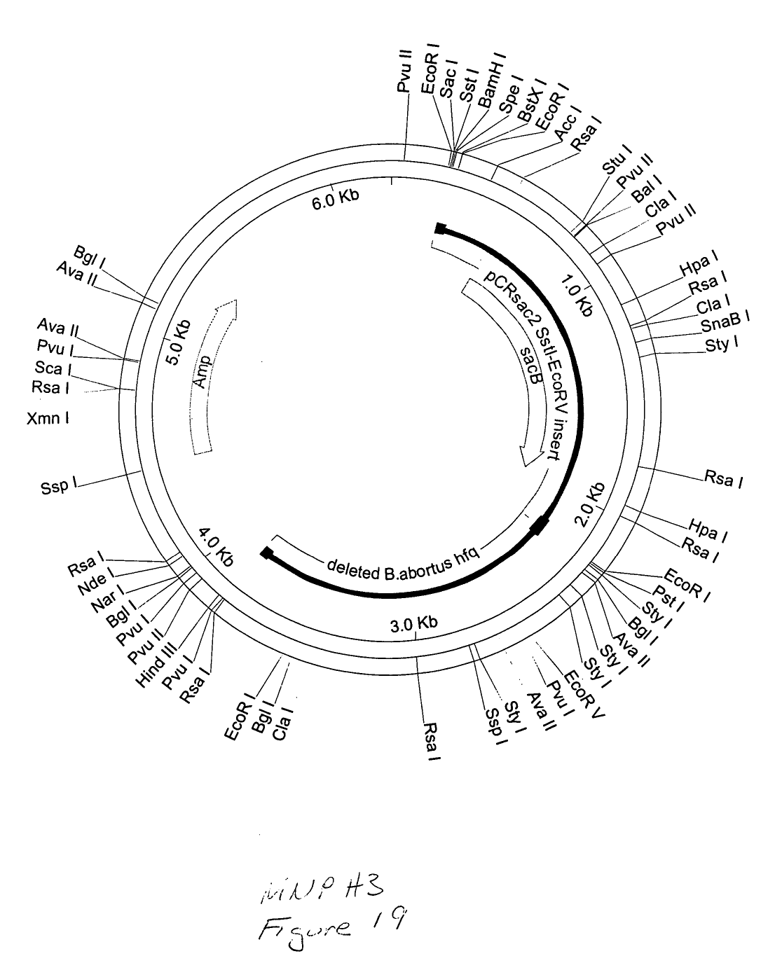

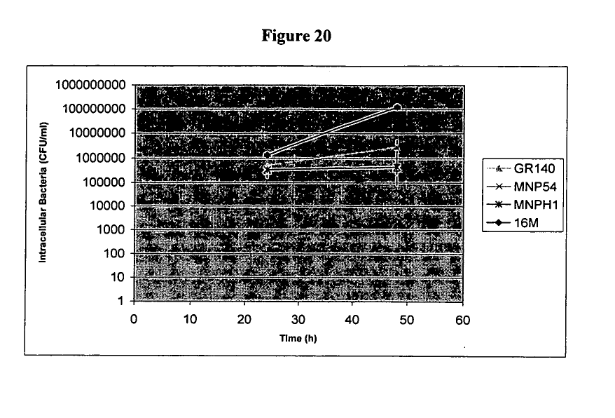

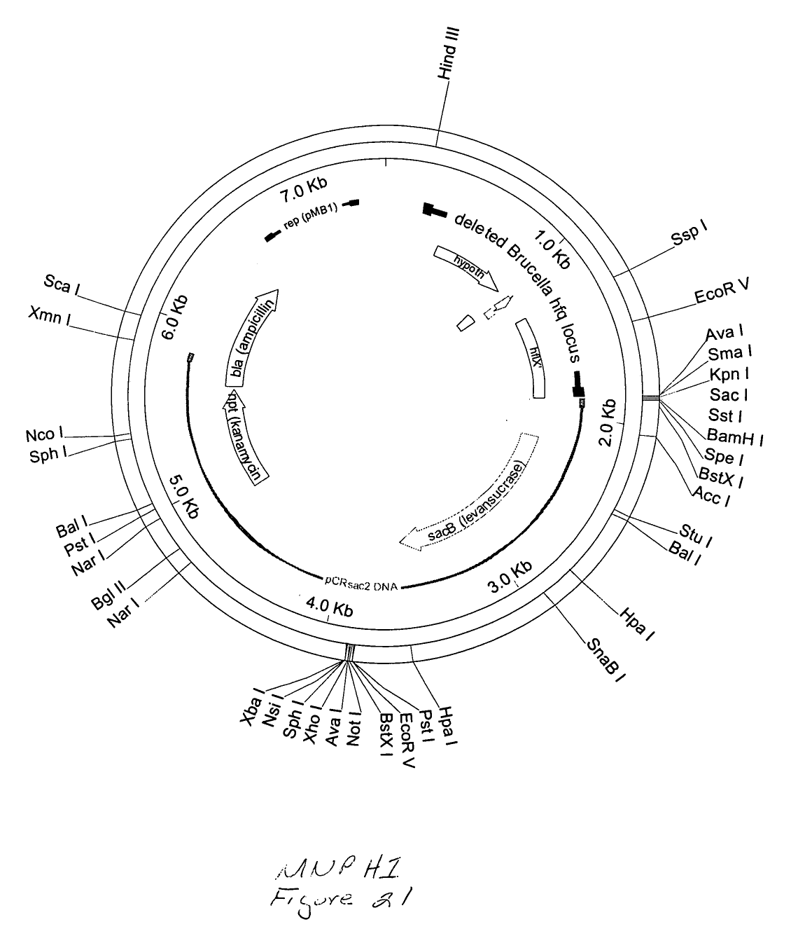

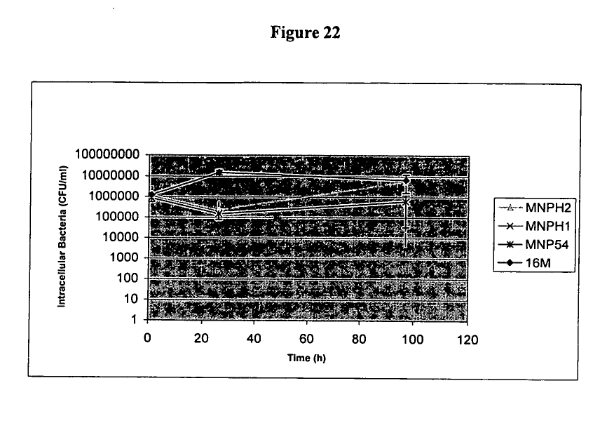

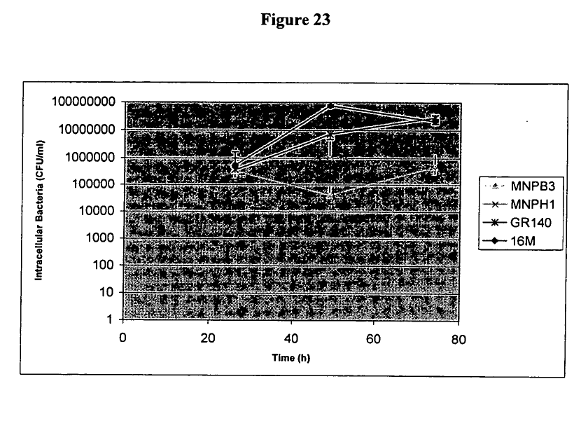

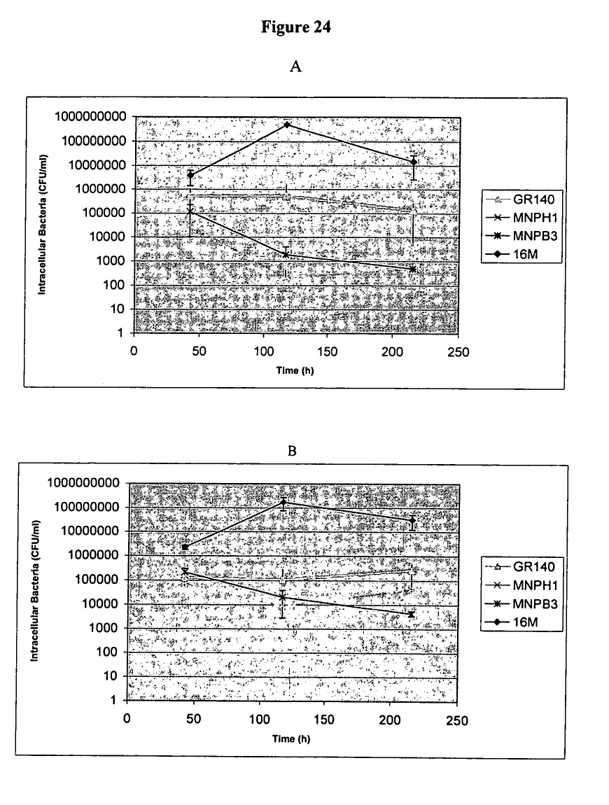

Interestingly, inguinal and axillary nodes became more reactive after challenge. Only one of four vaccinated monkeys developed bacteremia with 16M. The monkey remained positive over four consecutive sample times (11 days). In contrast, all four nonvaccinated animals developed bacteremia, with a duration ranging from 19 to 29 days (FIGS. 9 and 10) (P=0.017, Kruskal-Wallis test).

At necropsy 6 weeks after challenge, 16M was recovered from lymph nodes from all animals. The mean number of bacterial CFU in the infected nodes was lower in vaccinated animals than in nonvaccinated animals. In nonvaccinated animals, 16M was also recovered from liver, lung, spleen, bone marrow, epididymis, brain, bile, kidney, urine, and the subcutaneously implanted temperature recorder. In contrast, among vaccinated monkeys, these extranodal sites were uniformly negative for 16M, with the exception of the bacteremic animal, which had 700 CFU/g of 16M in its spleen. In addition, MNPH1 was recovered in broth, but not on plates, from one mediastinal lymph node from one vaccinated animal. All other Brucella isolates were confirmed to be 16M, not MNPH1. Two of four nonvaccinated, but none of the four vaccinated, 16M-challenged animals developed serum alkaline phosphatase values that were more than twice baseline. The increase in serum alkaline phosphatase levels is similar to results noted after oral administration of 16M in study WR9901.

These data indicate that MNPH1 administered at a dose of 1-2×10¹¹ CFU is immunogenic in Rhesus macaques. In addition, oral inoculation with 2×10¹¹ CFU of MNPH1 has a potent anti-Brucella effect, with significant reduction in bacteremia and prevention of fever. In addition, infection of nonlymphoid organs was prevented and the intensity of infection in lymph nodes was reduced. These data also demonstrated that the vaccine strain had nearly cleared, remaining at a barely detectable level in one lymph node of one animal 15 weeks after inoculation.

We tested safety and efficacy of a two-dose schedule of 10¹⁰CFU of MNPH1 in nonhuman primates. Six adult male Rhesus macaques were given two doses of MNPH1 orally by gavage 4 weeks apart. MNPH1 Lot No. 15 was manufactured and stored in 5% sucrose in TSB on Jul. 18, 2002. Bacteria were thawed and adjusted to approximate concentration using OD600 and suspended in phosphate-buffered saline (PBS) with sodium bicarbonate and administered by oral gavage. The actual inocula were confirmed by plate count to be 0.85×10¹⁰ CFU for the first dose and 0.97×10¹⁰ CFU for the second. Animals were followed for 56 days with serial blood sampling and clinical observation. Animals appeared clinically well and had minimal enlargement of the lymph nodes. The only enlargement was observed in the axillary lymph nodes, and no enlargement was greater than +1 on a scale of 0-4. The majority was +/−, which is barely palpable. The vaccinated animals continued to gain weight during the periods following gavage. The spleens also had minimal enlargement that had disappeared by 4 weeks following the second dose. During the period from the first dose of gavage to 4 weeks following the second dose (56 days), only one animal had bacteremia at a single time point of 18 days post-first dose and that was less than 1 CFU/ml recovered from broth culture. At no time was any Brucella recovered from the feces in any of the animals. One animal had anesthetic complications during sampling at 11 days following the first dose. A necropsy of this animal showed no remarkable changes and there was no evidence of acute brucellosis caused by MNPH1. Cultures of tissues from this animal demonstrated 81,250 CFU/g in the mesenteric lymph nodes, 200 CFU/g in the spleen, and 6 CFU/g in the liver. No MNPH1 was recovered from other tissues.

These data indicate that MNPH1 administered at a dose of 1-2×10¹¹ CFU is immunogenic in Rhesus macaques. In addition, oral immunization with 2×10¹¹ CFU of MNPH1 has a potent anti-Brucella effect, with significant reduction in bacteremia and prevention of fever. In addition, infection of nonlymphoid organs was prevented and the intensity of infection in lymph nodes was reduced. These data also demonstrated that the vaccine strain had nearly completely cleared, remaining at a barely detectable level in one lymph node of one animal 15 weeks after inoculation. The ongoing study IO07-03 indicates that further dose reduction, to 10¹⁰ CFU orally, leads to infection of the mononuclear phagocyte system (one animal with transient bacteremia and another with bacteria in lymph nodes, spleen and liver 11 days after oral vaccination) but is associated with minimal lymphadenopathy and splenic enlargement. In addition, the use of fermented, frozen bacteria for this study demonstrates that bacteria prepared by the proposed manufacturing process remained approximately 90% viable for at least a year and was infectious.

### Example 4

### Vaccine Stability and Release Testing

Stability

A research lot of strain MNPH1 was grown on TSB medium in a 2.3 liter fermenter and concentrated by centrifugation. The cells were resuspended in 90 ml of TSB with 5% sucrose and aliquoted into 10 ml glass vials (2 ml per vial). These vials were stored at −80° C. and sampled every 2-3 weeks over a period of 12 months. Results for these tests were 5.0±1.5×10¹⁰ CFUs/ml, confirming the long-term viability of the mutant (Table 11).

Technical Specification Sheet

Product: Brucella melitensis (MNPH1)-Live Vaccine Strain (Final Product)
Description: B. melitensis Live, attenuated Vaccine Strain
Fill Volume: By weight
Concentration: ≧10⁹ CFU/ml
Container Vial: TBD
Stopper: TBD
Cap: TBD
Container Size: 1 ml

Storage Conditions: −10 to −30° C.

TABLE 11
Proposed Lot Release and Characterization Assays
Testing for Oral Live, Attenuated MNPH1 Vaccine

| Test | Method | Specification |
|---|---|---|
| Potency | Enumerate CFUs | Mean viable count ≧10⁹ CFU/ml |
| Culture Characterization | Brucella agar or trypticase soy | Translucent colonies, convex, with an |
| | agar | entire edge and a smooth, shiny surface |
| | Colony Morphology | 0.5 to 0.7 by 0.6 to 1.5 μm gram- |
| | Gram Stain | negative cocci, coccobacilli, or short |
| | | rods after 5 days growth on Brucella |
| | | agar |
| Microbial ID (Identity) | Fatty Acid Methyl Ester | Profile according to B. melitensis |
| | (FAME) Analysis | as per FAME software |
| | Restriction Fragment Length | Profile according to B. melitensis |
| | Polymorphism (RFLP) | MNPH1 (100% match) |
| | Kanamycin disk identification | Resistance |
| | test | Report as tested |
| | PCR with deletion flanking | |
| | primers | |
| Microbial Limits Test | MLT | Absence of contaminating organisms |
| (MLT) (Purity) | | |
| pH | Measure pH of liquid vaccine | Report as tested |
| | using a standardized pH meter | |
| Viability Percentage | OD 600 versus CFUs | >90% predicted |
| General Safety | 21 CFR 610.11* | A safety test is satisfactory if all animals |
| | | meet all of the following requirements: |
| | | (1) They survive the test period. |
| | | (2) They do not exhibit any response |
| | | which is not specific for or |
| | | expected from the product and |
| | | which may indicate a difference |
| | | in its quality. |
| | | (3) They weigh no less at the end of |
| | | the test period than at the time of |
| | | injection. |
| Appearance of Product | Visual inspection of vials | To be determined |

Characterization Assays

| | | |
|---|---|---|
| Attenuation (Residual | Balb/c model | Recovery of less than 2 CFU of MNPH1 |
| Virulence) | | in spleen, inguinal lymph nodes, or |
| | | combined testis and epididymis in at |
| | | least 19 of 20 mice 8 weeks after |
| | | intraperitoneal (i.p.) injection of 1 × 10⁵ |
| | | CFUs. |
| Potency | Immunogenicity - anti-LPS | Report as tested |
| | antibodies by ELISA (8 weeks | |
| | after i.p. immunization) | |

The potency assay consists of administering 1×10⁵ CFU of MNPH1 vaccine i.p to 25, 6-8 week old male BALB/c mice. At 8 weeks postvaccination, collect heart blood to determine serum anti-LPS antibody by Enzyme-Linked Immunosorbent Assay (ELISA). Remove spleen, inguinal lymph nodes, testes, and epididymes, grind in tissue grinder, and suspend in 1 ml of medium. Culture one-half the final volume on Brucella agar to determine presence of vaccine strain.

### Example 5

### DNA Sequences of the Deleted purEK Locus, Deleted hfq Locus, Plasmid pMNH1 and plasmid pMNH54

The following is the plasmid pMNP54 with the deleted purEK locus. The DNA sequence consists of 1799 base pairs (linear). The deleted purEK locus was cloned in pMNP54 (pos. 251-2049), subsequently crossed into B. melitensis 16M genome to replace locus in strain MNP54. The deleted 229 base pairs replaced with 6 base pairs (pos. 442-447) containing introduced BglII. The sequence is labeled SEQ ID NO:13.

caagcttcaggaaatggaagatcaggtcattcctgacatcattgcctgaa
cattcaagaacactatagggaagagccggg
gttcgcccggctttgtcttttcaagaccttcctcatgccaaccgaaaagc
caagcaggaaagaccgatgagcgttgatgt
cgccattatcatgggaagccagtccgattgggaaaccatgcaccatgcag
ccgacacattggaggcgctcggcatctcct
tcgacgcacggatcgtttccgcccatcgcacccctgacaggctggtcgcc
ttcgccaagggggcgaaagcggaaggcttc
aaggtcatcatcgcaggcgccggcggcgcggcccacctgcccggcatggc
cgctgccatgacaccgcttcccgtctttgg
cgttccagttcaatccaaggcgctttcgggccaggattcgcagatctaag
cccggctccaccatcggcattatcggcggg
ggccagcttggccgtatgctcgccatggcagcggcgcgcttcggttatga
aaccataatccttgagccgcaggccggttg
cccggcagcacaggttgccaatcgccagattgtcgccgcctatgatgacc
cgaaggcgctggccgaacttgccgccgctt
ccgacgtcatcacctatgaatttgaaaatgtgccagtcagcgccgccgac
aagctggctgaaacggcgcttgttctgccc
ccgcccgccgcactggaaatctctcaggaccgcttcacagaaaagcagtt
tctcaacgaaagcggcattgaaaccgcgcc
ctggcggctcgtggatgacgaggaaacgctcatcgccgcgctcggcgcac
tgggcgggcgtggcatcctcaagatacggc
gtctgggttatgacggcaaggggcaggtgcgccttgcctccctcgatgaa
acccaggcctgcaacgcttttgcagccatc
aacaaggcgcctgcgattctcgaaggcttcgtggaattcgagcgcgaagt
ctccgtcatcgccgcgcgcgatcgcagcgg
caatgtcgccatcttcgatcttgcggaaaacgtccacaaggatggcattc
tcgccacgtccacagtgcctgccgcgatca
gcgtacagacggcagaagccgcgcgcacagccgccgaaaaactgttgcac
gcgctggactatgtcggtgtgctggggctt
gaatcttcgtgctgaaggacggcacgctgctcgccaatgaatttgccccg
cgtgtgcataattcgggccactggacgga
agcagcctgcgccatttcccaatttgagcagcatatccgcgctgtggcg
ggactgccgctcggcaatacggatcgccata
gcgactgtgtgatggaaaacctgattggcgacgatatcgaaaaggttcc
ggcgattctctgcgagaagaacgccgtgctg
catctttacggtaaaaaggaagctcgcgcgggccggaaaataggccatg
tgacccgcataaagccccgcacaatttaagc
tgcgccgggaatctgcacgattcccggcccttcctggccgcccgccaag
aaattcggggcctcggaccctgaatctgcgg
cgcgggagttgacatttgcctgaaaccttgtgtatttcggccaaccctt
cgggcacctgaccgtgcctgtaatcaattgg
cgccttgggcgcctgtttatgacgcccggcggcatcttcatgcgatggg
ccaaccagaccggtgattgacatgaaga
tcaagaactcgctcaaagccctcaaggcccgtcatcgcg

The following is plasmid pMNH1 with the deleted hfq locus. The DNA sequence contains 1633 base pairs (linear). Deleted hfq locus on pMNH1(pos. 234-1866), subsequently crossed into B. melitensis 16M genome to replace hfq locus in strains MNPH2, MNPH4. Deleted 327 base pairs of locus after position 811 (1044 in pMNH1). The sequence is labeled SEQ ID NO:14.

aAGCTTTTTGCCTATGCCCCGATCGAGAACGTACCGCCGGGTGTCGAA
CTGCATGATGCCGAAAGCATTTTGCCTGAAAC
GGCATTCAAGCGGCTTGATCCGGCCTATCCGAATTTTCATACCCAACT
GACTGTTGTTCAATTCAGTGATATTTTCCGTA
TCATGCTGATtAAATACCAGCAGGGCGTCTGGCTGGATACGGATGTCT
ATCTTCTCAAGCAGTTTCATCCCGATCCGGAC
AAGCCATACCTCGCCAGGGAGAATCGCTCCCGTGTCGGTGTGTCTGCG
CTTTATCTTCCGCATGACCATCCGATTATTCA
TGAATTCGAGGATTACATGGCCGCGACCGATCCGCCGCCGACCTGGCT
GGGTCTGCGCAGAGGCAAATTGCGTCCGCTTT
ACTATCGATTGATCCACAAGGAAGTAACGCCCGCCTCCATTGGCATTA
CAGTCTTTGGCAATGACGGTATCTCACGCCTC
GCCCGCAAATACGGGATTTTCAAGGATGCCGCGCCGCAGGAAAATTTC
TATTACTGGGTCGGCAAGGAGGCCACGCGCAT
CTATGATCCGGCCTATGGCCTGACGCCGATCCATCATCCGGAGTTTAT
CGGTTTTCATATCCATAAAAAGCATAAGGAAG
TCGTGTCGTTCCAGCCGGGAAGTTTTTACATGTGGGCAATAGATCGGG
TGCGCCCATTGCTGGAATCTAAAGAAAACAAA
GAGCTTGCGCTGGCAGATTGAATCCCGTGGCCTTCGCTGTATGTCAAA
TTTGCTACGAGCCAATGAAATTGCCGGTTGCG
CAAAGATTCCCCTATTTCGACAATCATGCCGAGCCAGCCTGTACAGAT
GTTTGAAGGCGAGGAAGCCTGACGCTTTCCGA
TATTTGCCGGGGCGTGGTAAGTATCCCGCCCCAGCTTTTGTTTGAGTAT
GTTTCGGCCAGAAAGCGCATCGCTATCGGGC
ATTGCTTGAACGTTCCGGGGCGTCTTCATTTGAAACAGCTTACGCGGT
GAtTTATCTGCAAGGCAGATCCATGCGTTCCC
GCGTCCCCGGAGAATATTACTTGTCCAAACCCAAGGATAAAAAAGCG
GCATCCCTCGATGGCAGCAAGGGCTTTGCCCTT
TCCGAGCCTaAGCCAACGAGAGCAGCCGTCATTGTTCCCGTCCTGCCG
GACCGTTATAATAGCGGCTCTGCGGGCGAGGA
TGGCGCAAGATCGCAGTTCCAGCGTTCCAACGAGGCGCGCCTTGAGG
AAGCGATCGGGCTTGCGCGCGCAATTGgTCTTG
ATATCGAACATGCGGAAATCGCCATCGTCACCAATCCGCGGCCCGCGA
CGCTGCTTGGTGCGGGCAAGGCCCAATCCATT
GCCGAAGTCGTCAAGGAAAAGGCGATTGGACTGGTGGTTGTCGATCAT
GCCCTGACGCCGGTGCAGCAGCGCAATCTTGA
AAAGGAATGGAACGCCAAGGTCATCGACCGAACCGGGCTCATTCTGG
AGATTTTCGGTGAACGCGCGCGCACCAAGGAAG
GCGCGCTTCAGGTGGAGCTGGCGCATCTCAACTACCAGAAGGGACGC
CTTGTCAGAAGCTGGACCCACCTTGAACGCCAG
CGCGGCGGTGGGGGCTTCCTTGGCGGCCCgggt

The following is plasmid pMNH1, having 7448 base pairs in the complete DNA sequence (circular). HindIII-SstI inserted into same of pCRsac2 [pEX100T sacU/sacL PCR in pCR2.1-TOPO:orient'n 2 or ‘2-2’; verified]. Now B. mel seq. The sequence is labeled SEQ ID NO:15.

AGCGCCCAATACGCAAACCGCCTCTCCCCGCGCGTTGGCCGATTCATT
AATGCAGCTGGCACGACAGGTTTCCCGACTGG
AAAGCGGGCAGTGAGCGCAACGCAATTAATGTGAGTTAGCTCACTCAT
TAGGCACCCCAGGCTTTACACTTTATGCTTCC
GGCTCGTATGTTGTGTGGAATTGTGAGCGGATAACAATTTCACACAGG
AAACAGCTATGACCATGATTACGCCaAGCTTT
TTGCCTATGCCCCGATCGAGAACGTACCGCCGGGTGTCGAACTGCATG
ATGCCGAAAGCATTTTGCCTGAAACGGCATTC
AAGCGGCTTGATCCGGCCTATCCGAATTTTCATACCCAACTGACTGTT
GTTCAATTCAGTGATATTTTCCGTATCATGCT
GATtAAATACCAGCAGGGCGTCTGGCTGGATACGGATGTCTATCTTCTC
AAGCAGTTTCATCCCGATCCGGACAAGCCAT
ACCTCGCCAGGGAGAATCGCTCCCGTGTCGGTGTGTCTGCGCTTTATCT
TCCGCATGACCATCCGATTATTCATGAATTC
GAGGATTACATGGCCGCGACCGATCCGCCGCCGACCTGGCTGGGTCTG
CGCAGAGGCAAATTGCGTCCGCTTTACTATCG
ATTGATCCACAAGGAAGTAACGCCCGCCTCCATTGGCATTACAGTCTT
TGGCAATGACGGTATCTCACGCCTCGCCCGCA
AATACGGGATTTTCAAGGATGCCGCGCCGCAGGAAAATTTCTATTACT
GGGTCGGCAAGGAGGCCACGCGCATCTATGAT
CCGGCCTATGGCCTGACGCCGATCCATCATCCGGAGTTTATCGGTTTTC
ATATCCATAAAAAGCATAAGGAAGTCGTGTC
GTTCCAGCCGGGAAGTTTTTACATGTGGGCAATAGATCGGGTGCGCCC
ATTGCTGGAATCTAAAGAAAACAAAGAGCTTG
CGCTGGCAGATTGAATCCCGTGGCCTTCGCTGTATGTCAAATTTGCTAC
GAGCCAATGAAATTGCCGGTTGCGCAAAGAT
TCCCCTATTTCGACAATCATGCCGAGCCAGCCTGTACAGATGTTTGAA
GGCGAGGAAGCCTGACGCTTTCCGATATTTGC
CGGGGCGTGGTAAGTATCCCGCCCCAGCTTTTGTTTGAGTATGTTTCGG
CCAGAAAGCGCATCGCTATCGGGCATTGCTT
GAACGTTCCGGGGCGTCTTCATTTGAAACAGCTTACGCGGTGAtTTATC
TGCAAGGCAGATCCATGCGTTCCCGCGTCCC
CGGAGAATATTACTTGTCCAAACCCAAGGATAAAAAAGCGGCATCCCT
CGATGGCAGCAAGGGCTTTGCCCTTTICCGAGC
CTaAGCCAACGAGAGCAGCCGTCATTGTTCCCGTCCTGCCGGACCGTT
ATAATAGCGGCTCTGCGGGCGAGGATGGCGCA
AGATCGCAGTTCCAGCGTTCCAACGAGGCGCGCCTTGAGGAAGCGAT
CGGGCTTGCGCGCGCAATTGgTCTTGATATCGA
ACATGCGGAAATCGCCATCGTCACCAATCCGCGGCCCGCGACGCTGCT
TGGTGCGGGCAAGGCCCAATCCATTGCCGAAG
TCGTCAAGGAAAAGGCGATTGGACTGGTGGTTGTCGATCATGCCCTGA
CGCCGGTGCAGCAGCGCAATCTTGAAAAGGAA
TGGAACGCCAAGGTCATCGACCGAACCGGGCTCATTCTGGAGATTTTC
GGTGAACGCGCGCGCACCAAGGAAGGCGCGCT
TCAGGTGGAGCTGGCGCATCTCAACTACCAGAAGGGACGCCTTGTCAG
AAGCTGGACCCACCTTGAACGCCAGCGCGGCG
GTGGGGGCTTCCTTGGCGGCCCgggtaccgagctcGGATCCACTAGTA
ACGGCCGCCAGTGTGCTGGAATTCGCCCTTAA
AGAGGAAAATAGACCAGTTGCAATCCAAACGAGAGTCTAATAGAATG
AGGTCGAAAAGTAAATCGCGCGGGTTTGTTACT
GATAAAGCAGGCAAGACCTAAAATGTGTAAAGGGCAAAGTGTATACT
TTGGCGTCACCCCTTACATATTTTAGGTCTTTT
TTTATTGTGCGTAACTAACTTGCCATCTTCAAACAGGAGGGCTGGAAG
AAGCAGACCGCTAACACAGTACATAAAAAAGG
AGACATGAACGATGAACATCAAAAAGTTTGCAAAACAAGCAACAGTA
TTAACCTTTACTACCGCACTGCTGGCAGGAGGC
GCAACTCAAGCGTTTGCGAAAGAAACGAACCAAAAGCCATATAAGGA
AACATACGGCATTTCCCATATTACACGCCATGA
TATGCTGCAAATCCCTGAACAGCAAAAAAATGAAAAATATCAAGTTCC
TGAGTTCGATTCGTCCACAATTAAAAATATCT
CTTCTGCAAAAGGCCTGGACGTTTGGGACAGCTGGCCATTACAAAACG
CTGACGGCACTGTCGCAAACTATCACGGCTAC
CACATCGTCTTTGCATTAGCCGGAGATCCTAAAAATGCGGATGACACA
TCGATTTACATGTTCTATCAAAAAGTCGGCGA
AACTTCTATTTGACAGCTGGAAAAACGCTGGCCGCGTCTTTAAAGACAG
CGACAAATTCGATGCAAATGATTCTATCCTAA
AAGACCAAACACAAGAATGGTCAGGTTCAGCCACATTTACATCTGACG
GAAAAATCCGTTTATTCTACACTGATTTCTCC
GGTAAACATTACGGCAAACAAACACTGACAACTGCACAAGTTAACGT
ATCAGCATCAGACAGCTCTTTGAACATCAACGG
TGTAGAGGATTATAAATCAATCTTTGACGGTGACGGAAAAACGTATCA
AAATGTACAGCAGTTCATCGATGAAGGCAACT
ACAGCTCAGGCGACAACCATACGCTGAGAGATCCTCACTACGTAGAA
GATAAAGGCCACAAATACTTAGTATTTGAAGCA
AACACTGGAACTGAAGATGGCTACCAAGGCGAAGAATCTTTATTTAAC
AAAGCATACTATGGCAAAAGCACATCATTCTT
CCGTCAAGAAAGTCAAAAACTTCTGCAAAGCGATAAAAAACGCACGG
CTGAGTTAGCAAACGGCGCTCTCGGTATGATTG
AGCTAAACGATGATTACACACTGAAAAAAGTGATGAAACCGCTGATT
GCATCTAACACAGTAACAGATGAAATTGAACGC
GCGAACGTCTTTAAAATGAACGGCAAATGGTATCTGTTCACTGACTCC
CGCGGATCAAAAATGACGATTGACGGCATTAC
GTCTAACGATATTTACATGCTTGGTTATGTTTCTAATTCTTTAACTGGC
CCATACAAGCCGCTGAACAAAACTGGCCTTG
TGTTAAAAATGGATCTTGATCCTAACGATGTAACCTTTACTTACTCACA
CTTCGCTGTACCTCAAGCGAAAGGAAACAAT
GTCGTGATTACAAGCTATATGACAAACAGAGGATTCTACGCAGACAA
ACAATCAACGTTTGCGCCTAGCTTCCTGCTGAA
CATCAAAGGCAAGAAAACATCTGTTGTCAAAGACAGCATCCTTGAAC
AAGGACAATTAACAGTTAACAAATAAAAACGCA
AAAGAAAATGCCGATTATGGTGCACTCTCAGTACAATCTGCTCTGATG
CCGCATAGTTAAGCCAGCCCCGACACCCGCCA
ACACCCGCTGACGCGCCCTGACGGGCTTGTCTGCTCCCGGCATCCGCT
TACAGACAAGCTGTGACCGTCTCCGGGAGCTG
CATGTGTCAGAGGTTTTCACCGTCATCACCGAAACGCGCGAGACGAAA
GGGCCTCGTGATACGCCTATTTTTATAAGGGC
GAATTCTGCAGATATCCATCACACTGGCGGCCGCTCGAGCATGCATCT
AGAGGGCCCAATTCGCCCTATAGTGAGTCGTA
TTACAATTCACTGGCCGTCGTTTTACAACGTCGTGACTGGGAAAACCC
TGGCGTTACCCAACTTAATCGCCTTGCAGCAC
ATCCCCCTTTCGCCAGCTGGCGTAATAGCGAAGAGGCCCGCACCGATC
GCCCTTCCCAACAGTTGCGCAGCCTGAATGGC
GAATGGGACGCGCCCTGTAGCGGCGCATTAAGCGCGGCGGGTGTGGT
GGTTACGCGCAGCGTGACCGCTACACTTGCCAG
CGCCCTAGCGCCCGCTCCTTTCGCTTTCTTCCCTTCCTTTCTCGCCACGT
TCGCCGGCTTTCCCCGTCAAGCTCTAAATC
GGGGGCTCCCTTTAGGGTTCCGATTTAGAGCTTTACGGCACCTCGACC
GCAAAAAACTTGATTTGGGTGATGGTTCACGT
AGTGGGCCATCGCCCTGATAGACGGTTTTTCGCCCTTTGACGTTGGAG
TCCACGTTCTTTAATAGTGGACTCTTGTTCCA
AACTGGAACAACACTCAACCCTATCGCGGTCTATTCTTTTGATTTATAA
GGGATTTTGCCGATTTCGGCCTATTGGTTAA
AAAATGAGCTGATTTAACAAATTCAGGGCGCAAGGGCTGCTAAAGGA
ACCGGAACACGTAGAAAGCCAGTCCGCAGAAAC
GGTGCTGACCCCGGATGAATGTCAGCTACTGGGCTATCTGGACAAGGG
AAAACGCAAGCGCAAAGAGAAAGCAGGTAGCT
TGCAGTGGGCTTACATGGCGATAGCTAGACTGGGCGGTTTTATGGACA
GCAAGCGAACCGGAATTGCCAGCTGGGGCGCC
CTCTGGTAAGGTTGGGAAGCCCTGCAAAGTAAACTGGATGGCTTTCTT
GCCGCCAAGGATCTGATGGCGCAGGGGATCAA
GATCTGATCAAGAGACAGGATGAGGATCGTTTCGCATGATTGAACAA
GATGGATTGCACGCAGGTTCTCCGGCCGCTTGG
GTGGAGAGGCTATTCGGCTATGACTGGGCACAACAGACAATCGGCTG
CTCTGATGCCGCCGTGTTCCGGCTGTCAGCGCA
GGGGCGCCCGGTTCTTTTTGTCAAGACCGACCTGTCCGGTGCCCTGAA
TGAACTGCAGGACGAGGCAGCGCGGCTATCGT
GGCTGGCCACGACGGGCGTTCCTTGCGCAGCTGTGCTCGACGTTGTCA
CTGAAGCGGGAAGGGACTGGCTGCTATTGGGC
GAAGTGCCGGGGCAGGATCTCCTGTCATCTCGCCTTGCTCCTGCCGAG
AAAGTATCCATCATGGCTGATGCAATGCGGCG
GCTGCATACGCTTGATCCGGCTACCTGCCCATTCGACCACCAAGCGAA
ACATCGCATCGAGCGAGCACGTACTCGGATGG
AAGCCGGTCTTGTCGATCAGGATGATCTGGACGAAGAGCATCAGGGG
CTCGCGCCAGCCGAACTGTTCGCCAGGCTCAAG
GCGCGCATGCCCGACGGCGAGGATCTCGTCGTGATCCATGGCGATGCC
TGCTTGCCGAATATCATGGTGGAAAATGGCCG
CTTTTCTGGATTCAACGACTGTGGCCGGCTGGGTGTGGCGGACCGCTA
TCAGGACATAGCGTTGGATACCCGTGATATTG
CTGAAGAGCTTGGCGGCGAATGGGCTGACCGCTTCCTCGTGCTTTACG
GTATCGCCGCTCCCGATTCGCAGCGCATCGCC
TTCTATCGCCTTCTTGACGAGTTCTTCTGAATTGAAAAAGGAAGAGTA
TGAGTATTCAACATTTCCGTGTCGCCCTTATT
CCCTTTTTTGCGGCATTTTGCCTTCCTGTTTTTGCTCACCCAGAAACGCT
GGTGAAAGTAAAAGATGCTGAAGATCAGTT
GGGTGCACGAGTGGGTTACATCGAACTGGATCTCAACAGCGGTAAGA
TCCTTGAGAGTTTTCGCCCCGAAGAACGTTTTC
CAATGATGAGCACTTTTAAAGTTCTGCTATGTCATACACTATTATCCCG
TATTGACGCCGGGCAAGAGCAACTCGGTCGC
CGGGCGCGGTATTCTCAGAATGACTTGGTTGAGTACTCACCAGTCACA
GAAAAGCATCTTACGGATGGCATGACAGTAAG
AGAATTATGCAGTGCTGCCATAACCATGAGTGATAACACTGCGGCCAA
CTTACTTCTGACAACGATCGGAGGACCGAAGG
AGCTAACCGCTTTTTTGCACAACATGGGGGATCATGTAACTCGCCTTG
ATCGTTGGGAACCGGAGCTGAATGAAGCCATA
CCAAACGACGAGAGTGACACCACGATGCCTGTAGCAATGCCAACAAC
GTTGCGCAAACTATTAACTGGCGAACTACTTAC
TCTAGCTTCCCGGCAACAATTAATAGACTGGATGGAGGCGGATAAAGT
TGCAGGACCACTTCTGCGCTCGGCCCTTCCGG
CTGGCTGGTTTATTGCTGATAAATCTGGAGCCGGTGAGCGTGGGTCTC
GCGGTATCATTGCAGCACTGGGGCCAGATGGT
AAGCCCTCCCGTATCGTAGTTATCTACACGACGGGGAGTCAGGCAACT
ATGGATGAACGAAATAGACAGATCGCTGAGAT
AGGTGCCTCACTGATTAAGCATTGGTAACTGTCAGACCAAGTTTACTC
ATATATACTTTAGATTGATTTAAAACTTCATT
TTTAATTTAAAAGGATCTAGGTGAAGATCCTTTTTGATAATCTCATGAC
CAAAATCCCTTAACGTGAGTTTTCGTTCCAC
TGAGCGTCAGACCCCGTAGAAAAGATCAAAGGATCTTCTTGAGATCCT
TTTTTTCTGCGCGTAATCTGCTGCTTGCAAAC
AAAAAAACCACCGCTACCAGCGGTGGTTTGTTTGCCGGATCAAGAGCT
ACCAACTCTTTTTCCGAAGGTAACTGGCTTCA
GCAGAGCGCAGATACCAAATACTGTCCTTCTAGTGTAGCCGTAGTTAG
GCCACCACTTCAAGAACTCTGTAGCACCGCCT
ACATACCTCGCTCTGCTAATCCTGTTACCAGTGGCTGCTGCCAGTGGC
GATAAGTCGTGTCTTACCGGGTTGGACTCAAG
ACGATAGTTACCGGATAAGGCGCAGCGGTCGGGCTGAACGGGGGGTT
CGTGCACACAGCCCAGCTTGGAGCGAACGACCT
ACACCGAACTGAGATACCTACAGCGTGAGCATTGAGAAAGCGCCACG
CTTCCCGAAGGGAGAAAGGCGGACAGGTATCCG
GTAAGCGGCAGGGTCGGAACAGGAGAGCGCACGAGGGAGCTTCCAGG
GGGAAACGCCTGGTATCTTTATAGTCCTGTCGG
GTTTCGCCACCTCTGACTTGAGCGTCGATTTTTGTGATGCTCGTCAGGG
GGGCGGAGCCTATGGAAAAACGCCAGCAACG
CGGCCTTTTTACGGTTCCTGGCCTTTTGCTGGCCTTTTGCTCACATGTTC
TTTCCTGCGTTATCCCCTGATTCTGTGGAT
AACCGTATTACCGCCTTTGAGTGAGCTGATACCGCTCGCCGCAGCCGA
ACGACCGAGCGCAGCGAGTCAGTGAGCGAGGA
AGCGGAAG

The following is the DNA sequence of plasmid pMNP54-correct, containing 8931 base pairs (circular). Insertion of purE197 ‘puramp’ PCR into pJQ200SK SpeI. Insert=lower case/Bru: 251-2049. NOTE: thought in *KS*; probably switched in shipt. The sequence is labeled SEQ ID NO:16.

CTGGCGAAGGGGGGATGTGCTGCAAGGCGATTAAGTTGGGTAACGCC
AGGGTTTTCCCAGTCACGACGTTGTAAAACGAC
GGCCAGTGAATTGTAATACGACTCACTATAGGGCGAATTGGAGCTCCA
CCGCGGTGGCGGCCGCTCTAGAActagtaggc
tttacactttatgcttccggctcgtatgttgtgtggaattgtgagcggat
aacaatttcacacaggaaacagctatgaccatgattacgccaagcttcag
gaaatggaagatcaggtcattcctgacatcattgcctgaacattcaagaa
cactataggg
aagagccggggttcgcccggctttgtcttttcaagaccttcctcatgcca
accgaaaagccaagcaggaaagaccgatga
gcgttgatgtcgccattatcatgggaagccagtccgattgggaaaccatg
caccatgcagccgacacattggaggcgctc
ggcatctccttcgacgcacggatcgtttccgcccatcgcacccctgacag
gctggtcgccttcgccaagggggcgaaagc
ggaaggcttcaaggtcatcatcgcaggcgccggcggcgcggcccacctgc
ccggcatggccgctgccatgacaccgcttc
ccgtctttggcgttccagttcaatccaaggcgctttcgggccaggattcg
cagatctaagcccggctccaccatcggcat
tatcggcgggggccagcttggccgtatgctcgccatggcagcggcgcgct
tcggttatgaaaccataatccttgagccgc
aggccggttgcccggcagcacaggttgccaatcgccagattgtcgccgcc
tatgatgacccgaaggcgctggccgaactt
gccgccgcttccgacgtcatcacctatgaatttgaaaatgtgccagtcag
cgccgccgacaagctggctgaaacggcgct
tgttctgcccccgcccgccgcactggaaatctctcaggaccgcttcacag
aaaagcagtttctcaacgaaagcggcattg
aaaccgcgccctggcggctcgtggatgacgaggaaacgctcatcgccgcg
ctcggcgcactgggcgggcgtggcatcctc
aagatacggcgtctgggttatgacggcaaggggcaggtgcgccttgcctc
cctcgatgaaacccaggcctgcaacgcttt
tgcagccatcaacaaggcgcctgcgattctcgaaggcttcgtggaattcg
agcgcgaagtctccgtcatcgccgcgcgcg
atcgcagcggcaatgtcgccatcttcgatcttgcggaaaacgtccacaag
gatggcattctcgccacgtccacagtgcct
gccgcgatcagcgtacagacggcagaagccgcgcgcacagccgccgaaaa
actgttgcacgcgctggactatgtcggtgt
gctggggcttgaattcttcgtgctgaaggacggcacgctgctcgccaatg
aatttgccccgcgtgtgcataattcgggcc
actggacggaagcagcctgcgccatttcccaatttgagcagcatatccgc
gctgtggcgggactgccgctcggcaatacg
gatcgccatagcgactgtgtgatggaaaacctgattggcgacgatatcga
aaaggttccggcgattctctgcgagaagaa
cgccgtgctgcatcffacggtaaaaaggaagctcgcgcgggccggaaaat
aggccatgtgacccgcataaagccccgca
caatttaagctgcgccgggaatctgcacgattcccggcccttcctggccg
cccgccaagaaattcggggcctcggaccct
gaatctgcggcgcgggagttgacatttgcctgaaaccttgtgtatttcgg
ccaacccttcgggcacctgaccgtgcctgt
aatcaattggcgccttgggcgcctgtttttatgacgcccggcggcatctt
catgtcgatgggccaaccagaccggtgatt
gacatgaagatcaagaactcgctcaaagccctcaaggcccgtcatcgcgc
atgcctgcaggtcgactctagaggatcccc
gggtaccgagctcgaattcactggccgtcgttttacaacgtcgtgactgg
gaaaaccctggcgttacccaacttaatcgc
cttgcagcacatccccctttcgccagctggcgtaatagcgaagaggcccg
caccgatcgcccttcccaacagttgcgcag
cctgaatggcgaatggcgcctgatgcggtattttctccttacgcatctgt
gcggtatttcacaccgcatatggtgcactctcagtacaatctgctctgat
gccgcatagttaagccagccccgacacccgccaacacccgctgacgcgcc
ctgacgggct
tgtctgctcccggcatccgcttacagacaagctgtgaccgtctccgggag
ctgcattgtcagaggttttcaccgtcatc
accgaaacgcgcgagacgaaagggcctcgtgatacgcctatttataggtt
aatgtcatgataataatggtttcttagacgtcaggtggcacttttcgggg
aaatgtgcgcggaacccctatttgtttatttttctaaatacattcaaata
tgtatccgctcatgagacaataaccctgataaatgcttcaataatattga
aaaaggaagagtatgagtattcaacatttccgtgtcgcccttattccctt
ttttgcggcattttgccttcctgtttttgctcacccagaaacgctggtga
aagtaaaagatgctgaagatcagttgggtgcacgagtgggttacatcgaa
ctggatctcaacagcggtaagatccttgagagttttcgccccgaagaa
cgttttccaatgatgagcacttttaaagttctgctatgtggcgcggtatt
atcccgtattgacgccgggcaagagcaactcggtcgccgcatacactatt
ctcagaatgacttggttgagtactcaccagtcacagaaaagcatcuacgg
atggcatga
cagtaagagaattatgcagtgctgccataaccatgagtgataacactgcg
gccaacuacttctgacaacgatcggagga
ccgaaggagctaaccgcttttttgcacaacatgggggatcatgtaactcg
ccttgatcgttgggaaccggagctgaatga
agccataccaaacgacgagcgtgacaccacgatgcctgtagcaatggcaa
caacgttgcgcaaactattaactggcgaac
tacttactctagcttcccggcaacaattaatagactggatggaggcggat
aaagttgcaggaccacnctgcgctcggcc
cttccggctggctggtttattgctgataaatctggagccggtgagcgtgg
gtctcgcggtatcattgcagcactggggcc
agatggtaagccctcccgtatcgtagttatctacacgacggggagtcagg
caactatggatgaacgaaatagacagatcg
ctgagataggtgcctcactgataagcattggtaactgtcagaccaagttt
actcatatatactttagattgatttaaaacttcatttttaatttaaaagg
atctaggtgaagatcctttttgataatctcatgaccaaaatcccttaacg
tgagttttcgttccactgagcgtcagaccccgtagaaaagataCTAGTGG
ATCCCCCGGGCTGCAGGAATTCGATATCAAGCTTATCGA
TACCGTCGACCTCGAGGGGGGGCCCGGTACCCAGCTTTTGTTCCCTTT
AGTGAGGGTTAATTCCGAGCTTGGCGTAATCA
TGGTCATAGCTGTTTCCTGTGTGAAATTGTTATCCGCTCACAATTCCAC
ACAACATAGGAGCCGGAAGCATAAAGTGTAA
AGCCTGGGGTGCCTAATGAGTGAGGTAACTCACATTAATTGCGTTGCG
CTCACTGCCCGCTTTCCAGTCGGGAAACCTGT
CGTGCCAGCGGGAATTAATTCTGTCCCTCCTGTTCAGCTACTGACGGG
GTGGTGCGTAACGGCAAAAGCACCGCCGGACA
TCAGCGCTAGCGGAGTGTATACTGGCTTACTATGTTGGCACTGATGAG
GGTGTCAGTGAAGTGCTTCATGTGGCAGGAGA
AAAAAGGCTGCACCGGTGCGTCAGCAGAATATGTGATACAGGATATA
TTCCGCTTCCTCGCTCACTGACTCGCTACGCTC
GGTCGTTCGACTGCGGCGAGCGGAAATGGCTTACGAACGGGGCGGAG
ATTTCCTGGAAGATGCCAGGAAGATACTTAACA
GGGAAGTGAGAGGGCCGCGGCAAAGCCGTTTTTCCATAGGCTCCGCC
CCCCTGACAAGCATCACGAAATCTGACGCTCAA
ATCAGTGGTGGCGAAACCCGACAGGACTATAAAGATACCAGGCGTTT
CCCCTGGCGGCTCCCTCGTGCGCTCTCCTGTTC
CTGCCTTTCGGTTTACCGGTGTCATTCCGCTGTTATGGCCGCGTTTGTC
TCATTCCACGCCTGACACTCAGTTCCGGGTA
GGCAGTTCGCTCCAAGCTGGACTGTATGCACGAACCCCCCGTTCAGTT
CGACCGCTGCGCCTTATCCGGTAACTATCGTC
TTGAGTCCAACCCGGAAAGACATGCAAAAGCACCACTGGCAGCAGCC
ACTGGTAATTGATTTAGAGGAGTTAGTCTTGAA
GTCATGCGCCGGTTAAGGCTAAACTGAAAGGACAAGTTTTGGTGACTG
CGCTCCTCCAAGCCAGTTACCTCGGTTCAAAG
AGTTGGTAGCTCAGAGAACCTTCGAAAACCGCCCTGCAAGGCGGTTTT
TTCGTTTTCAGAGCAAGAGATTACGCGCAGAC
CAAAACGATCTCAAGAAGATCATCTTATTAATCAGATAAAATATTTCT
AGCTAGAGTCGATCTTCGCCAGCAGGGCAGGA
TCGTGGCATCACCGAACCGCGCCGTGCCGCTCGTCGGTGAGCCAGAGT
TTCAGCAGGCCGCCCAGggggAGGTCGCCATT
GATGCGGGCCAGCTCGCGGACGTGCTCATAGTCCACGACGCCCGTGAT
TTTGTAGCCCTGGCCGACGCGACAGGTAGGCC
GACAGCTCATGCCGGCCGCCGCCGCCTTTTCCTCAATCGCTCTTCGTTC
GTCTGGAAGGCAGTACACCTTGATAGGTGGG
CTGCCCTTCCTGGTTGGCTTGGTTTCATCAGCCATCCGCTTGCCCTCAT
CTGTTACGCCGGCGGTAGCCGGCCAGCCTCG
CAGAGCAGGATTCCCGTTGAGCACCGCCAGGTGCGAATAAGGGACAG
TGAAGAAGGAACACCCGCTCGCGGGTGGGCCTA
CTTCACCTATCCTGCCCGGCTGACGCCGTTGGATACACCAAGGAAAGT
CTACGCGAACCCTTTGGCAAAATCCTGTATAT
CGTGCGAAAAAGGATGGATATACCGAAAAAATCGCTATAATGACCCC
GAAGCAGGGTTATGCAGCGGAAAAGCGCTGCTT
CCCTGCTGTTTTGTGGAATATCTACCGACTGGAAACAGGCAAATGCAG
GAAATTACTGAACTGAGGGGACAGGCGAGAGA
CGATGCCAAAGAGCTACACCGACGAGCTGGCCGAGTGGGTTGAATCC
CGCGCGGCCAAGAAGCGCCGGCGTGATGAGGCT
GCGGTTGCGTTCCTGGCGGTGAGGGCGGATGTCGATCGACTCTAGCTA
GAGGATCGATCCTTTTTAACCCATCACATATA
CCTGCCGTTCACTATTATTTAGTGAAATGAGATATTATGATATTTTCTG
AATTGTGATTAAAAAGGCAACTTTATGCCCA
TGCAACAGAAACTATAAAAAATACAGAGAATGAAAAGAAACAGATAG
ATTTTTTAGTTCTTTAGGCCCGTAGTCTGCAAA
TCCTTTTATGATTTTCTATCAAACAAAAGAGGAAAATAGACCAGTTGC
AATCCAAACGAGAGTCTAATAGAATGAGGTCG
AAAAGTAAATCGCGCGGGTTTGTTACTGATAAAGCAGGCAAGACCTA
AAATGTGTAAAGGGCAAAGTGTATACTTTGGCG
TCACCCCTTACATATTTTAGGTCTTTTTTTATTGTGCGTAACTAACTTGC
CATCTTCAAACAGGAGGGCTGGAAGAAGCA
GACCGCTAACACAGTACATAAAAAAGGAGACATGAACGATGAACATC
AAAAAGTTTGCAAAACAAGCAACAGTATTAACC
TTTACTACCGCACTGCTGGCAGGAGGCGCAACTCAAGCGTTTGCGAAA
GAAACGAACCAAAAGCCATATAAGGAAACATA
CGGCATTTCCCATATTACACGCCATGATATGCTGCAAATCCCTGAACA
GCAAAAAAATGAAAAATATCAAGTTCCTGAAT
TCGATTCGTCCACAATTAAAAATATCTCTTCTGCAAAAGGCCTGGACG
TTTGGGACAGCTGGCCATTACAAAACGCTGAC
GGCACTGTCGCAAACTATCGCGGCTACCACATCGTCTTTGCATTAGCC
GGAGATCCTAAAAATGCGGATGACACATCGAT
TTACATGTTCTATCAAAAAGTCGGCGAAACTTCTATTGACAGCTGGAA
AAACGCTGGCCGCGTCTTTAAAGACAGCGACA
AATTCGATGCAAATGATTCTATCCTAAAAGACCAAACACAAGAATGGT
CAGGTTCAGCCACATTTACATCTGACGGAAAA
ATCCGTTTATTCTACACTGATTTCTCCGGTAAACATTACGGCAAACAA
ACACTGACAACTGCACAAGTTAACGTATCAGC
ATCAGACAGCTCTTTGAACATCAACGGTGTAGAGGATTATAAATCAAT
CTTTGACGGTGACGGAAAAACGTATCAAAATG
TACAGCAGTTCATCGATGAAGGCAACTACAGCTCAGGCGACAACCAT
ACGCTGAGAGATCCTCACTACGTAGAAGATAAA
GGCCACAAATACTTAGTATTTGAAGCAAACACTGGAACTGAAGATGG
CTACCAAGGCGAAGAATCTTTATTTAACAAAGC
ATACTATGGCAAAAGCACATCATTCTTCCGTCAAGAAAGTCAAAAACT
TCTGCAAAGCGATAAAAAACGCACGGCTGAGT
TAGCAAACGGCGCTCTCGGTATGATTGAGCTAAACGATGATTACACAC
TGAAAAAAGTGATGAAACCGCTGATTGCATCT
AACACAGTAACAGATGAAATTGAACGCGCGAACGTCTTTAAAATGAA
CGGCAAATGGTACCTGTTCACTGACTCCCGCGG
ATCAAAAATGACGATTGACGGCATTTCGTCTAACGATATTTACATGCT
TGGTTATGTTTCTAATTCTTFAACTGGCCCAT
ACAAGCCGCTGAACAAAACTGGCCTTGTGTTAAAAATGGATCTTGATC
CTAACGATGTAACCTTTACTTACTCACACTTC
GCTGTACCTCAAGCGAAAGGAAACAATGTCGTGGTGATTACAAGCTAT
ATGACAAACAGAGGATTCTACGCAGACAAACA
ATCAACGTTTGCGCCAAGCTTCCTGCTGAACATCAAAGGCAAGAAAAC
ATCTGTTGTCAAAGACAGCATCCTTGAACAAG
GACAATTAACAGTTAACAAATAAAAACGCAAAAGAAAATGCCGATGG
CCGCGGCGTTGTGACAATTTACCGAACAACTCC
GCGGCCGGGAAGCCGATCTCGGCTTGAACGAATTGTTAGGTGGCGGTA
CTTGGGTCGATATCAAAGTGCATCACTTCTTC
CCGTATGCCCAACTTTGTATAGAGAGCCACTGCGGGATCGTCACCGTA
ATCTGCTTGCACGTAGATCACATAAGCACCAA
GCGCGTTGGCCTCATGCTTGAGGAGATTGATGAGCGCGGTGGCAATGC
CCTGCCTCCGGTGCTCGCCGGAGACTGCGAGA
TCATAGATATAGATCTCACTACGCGGCTGCTCAAACCTGGGCAGAACG
TAAGCCGCGAGAGCGCCAACAACCGCTTCTTG
GTCGAAGGCAGCAAGCGCGATGAATGTCTTACTACGGAGCAAGTTCCC
GAGGTAATCGGAGTCCGGCTGATGTTGGGAGT
AGGTGGCTACGTCTCCGAACTCACTACCGAAAAGATCAAGAGCAGCC
CGCATGGATTTGACTTGGTCAGGGCCGAGCCTA
CATGTGCGAATGATGCCCATACTTGAGCCACCTAACTTTGTTTTAGGG
CGACTGCCCTGCTGCGTAACATCGTTGCTGCT
GCGTAACATCGTTGCTGCTCCATAACATGAAACATCGACCCACGGCGT
AACGCGCTTGCTGCTTGGATGCCCGAGGCATA
GACTGTACAAAAAAACAGTCATAACAAGCCATGAAAACCGCCACTGC
GCCGTTACCACCGCTGCGTTCGGTCAAGGTTCT
GGACCAGTTGCGTGAGCGCATACGCTACflGCATTACAGTTTACGAAC
CGAACAGGCTTATGTCAACTGGGTTCGTGCCT
TCATCCGTTTCCACGGTGTGCGTCACCCGGCAACCTTGGGCAGCAGCG
AAGTCGAGGCATTTCTGTCCTGGCTGGCGAAC
GAGCGCAAGGTTTCGGTCTCCACGCATCGTCAGGCATTGGCGGCCAAG
CTGTTCTTCTACGGCAAGGTGCTGTGCACGGA
TCTGCCCTGGCTTCAGGAGATCGGAAGACCTCGGCCGTCGCGGCGCTT
GCCGGTGGTGCTGACCCCGGATGAAGTGGTTC
GCATCCTCGGTTTTCTGGAAGGCGAGCATCGTTTGTTCGCCCAGCTTCT
GTATGGAACGGGCATGCGGATCAGTGGGTTT
GCAACTGCGGGTCAAGGTCTGGATTTCGATCACGGCACGATCATGCTG
CGGGAGGGCAAGGGCTCCAAGGATCGGGCCTT
GATGTTACCCGAGAGCTTGGCACCCACGCTGCGCGAGCAGAATTAATT
CCC

1. A Brucella melitensis strain that has a deletion mutation of the purEK gene, and which does not have any antibiotic resistance determinant gene at the purEK gene deletion site.

2. The Brucella melitensis strain of claim 1, which has a 16M genetic background, and wherein the deletion mutation of the purEK gene is located at nucleotides positions 309305 and 309532 in chromosome I.

3. The Brucella melitensis strain of claim 1, which is designated as MNP54.

4. The Brucella melitensis strain of claim 1, which further includes a deletion mutation of the hfq gene, and which has a kanamycin resistance determinant marker gene inserted in hfq gene deletion site.

5. The Brucella melitensis strain of claim 4, which has a 16M genetic background, and wherein the deletion mutation of the hfq gene is located between nucleotide positions 900261 and 900596 in chromosome I.

6. The Brucella melitensis strain of claim 4, which is designated as MNPH3.

7. The Brucella melitensis strain of claim 1, which further includes a deletion mutation of the hfq gene, and which does not have any antibiotic resistance determinant gene inserted in hfq gene deletion site.

8. The Brucella melitensis strain of claim 7, which is designated as MNPH4.

9. A Brucella melitensis strain that has a deletion mutation of the hfq gene, and which has a kanamycin resistance determinant marker gene inserted in hfq gene deletion site.

10. The Brucella melitensis strain of claim 9, which is designated as MNPH1.

11. A Brucella melitensis strain that has a deletion mutation of the hfq gene, and which does not have any antibiotic resistance gene inserted in hfq gene deletion site.

12. The Brucella melitensis strain of claim 11, which has a 16M genetic background, and wherein the deletion mutation of the hfq gene is located between nucleotide positions 900261 and 900596 on chromosome I.

13. The Brucella melitensis strain of claim 11, which is designated as MNPH2.

14. An immunogenic composition comprising a Brucella melitensis strain selected from the group consisting of:
(i) a Brucella melitensis strain that has a deletion mutation of the purEK gene, and which does not have any antibiotic resistance determinant gene in purEK gene deletion site;
(ii) a Brucella melitensis strain that has a deletion mutation of the hfq gene, and which has a kanamycin resistance determinant marker gene inserted in hfq gene deletion site;
(iii) a Brucella melitensis strain that has a deletion mutation of the hfq gene, and which does not have any antibiotic resistance determinant gene inserted in hfq gene deletion site;
(iv) a Brucella melitensis strain that has a deletion mutation of the purEK gene which does not have any antibiotic resistance determinant gene in purEK gene deletion site, and that has a deletion mutation of the hfq gene, wherein a kanamycin resistance determinant marker gene is inserted in hfq gene deletion site; and
(v) a Brucella melitensis strain that has a deletion mutation of the purEK gene which does not have any antibiotic resistance determinant gene in purEK gene deletion site, and that has a deletion mutation of the hfq gene, which does not have a kanamycin resistance determinant marker gene inserted in hfq gene deletion site,
and a pharmaceutically acceptable carrier.

15. The immunogenic composition of claim 15, wherein the Brucella melitensis strain of (i) is the strain designated as MNP54, the Brucella melitensis strain of (ii) is the strain designated as MNPH1, the Brucella melitensis strain of (iii) is the strain designated as MNPH2, the Brucella melitensis strain of (iv) is the strain designated as MNPH3, and the Brucella melitensis strain of (v) is the strain designated as MNPH4.

16. A veterinary vaccine against Brucella melitensis comprising a Brucella melitensis strain selected from the group consisting of:
(i) a Brucella melitensis strain that has a deletion mutation of the purEK gene, and which does not have any antibiotic resistance determinant gene in purEK gene deletion site;
(ii) a Brucella melitensis strain that has a deletion mutation of the hfq gene, and which has a kanamycin resistance determinant marker gene inserted in hfq gene deletion site;
(iii) a Brucella melitensis strain that has a deletion mutation of the hfq gene, and which does not have any antibiotic resistance determinant gene inserted in hfq gene deletion site;
(iv) a Brucella melitensis strain that has a deletion mutation of the purEK gene which does not have any antibiotic resistance determinant gene in purEK gene deletion site, and that has a deletion mutation of the hfq gene, wherein a kanamycin resistance determinant marker gene is inserted in hfq gene deletion site; and
(v) a Brucella melitensis strain that has a deletion mutation of the purEK gene which does not have any antibiotic resistance determinant gene in purEK gene deletion site, and that has a deletion mutation of the hfq gene, which does not have a kanamycin resistance determinant marker gene inserted in hfq gene deletion site,
and a pharmaceutically acceptable carrier.

17. The veterinary vaccine of claim 16, wherein the Brucella melitensis strain of (i) is the strain designated as MNP54, the Brucella melitensis strain of (ii) is the strain designated as MNPH1, the Brucella melitensis strain of (iii) is the strain designated as MNPH2, the Brucella melitensis strain of (iv) is the strain designated as MNPH3, and the Brucella melitensis strain of (v) is the strain designated as MNPH4.


SEQUENCE LISTING (16 sequences)

SEQ ID NO: 1 - DNA, 19 bp, Artificial Sequence
  Description of Artificial Sequence Synthetic Primer
acgcctcgcccgcaaatac

SEQ ID NO: 2 - DNA, 19 bp, Artificial Sequence
  Description of Artificial Sequence Synthetic Primer
cctcgccttcaaacatctg

SEQ ID NO: 3 - DNA, 22 bp, Artificial Sequence
  Description of Artificial Sequence Synthetic Primer
ataaaaataggcgtatcacgag

SEQ ID NO: 4 - DNA, 22 bp, Artificial Sequence
  Description of Artificial Sequence Synthetic Primer
aaagaggaaaatagaccagttg

SEQ ID NO: 5 - DNA, 27 bp, Artificial Sequence
  Description of Artificial Sequence Synthetic Primer
gactagtaggctttacactttatgctt

SEQ ID NO: 6 - DNA, 27 bp, Artificial Sequence
  Description of Artificial Sequence Synthetic Primer
cactagtatcttttctacggggtctga

SEQ ID NO: 7 - DNA, 18 bp, Artificial Sequence
  Description of Artificial Sequence Synthetic Primer
caccatgcagccgacaca

SEQ ID NO: 8 - DNA, 18 bp, Artificial Sequence
  Description of Artificial Sequence Synthetic Primer
cgcgccgcagattcaggg

SEQ ID NO: 9 - DNA, 18 bp, Artificial Sequence
  Description of Artificial Sequence Synthetic Primer
gagtgccgacgggaatac

SEQ ID NO: 10 - DNA, 18 bp, Artificial Sequence
  Description of Artificial Sequence Synthetic Primer
gatccggcgaggtagaaa

SEQ ID NO: 11 - DNA, 21 bp, Artificial Sequence
  Description of Artificial Sequence Synthetic Primer
ccgaaaagccaagcaggaaag

SEQ ID NO: 12 - DNA, 21 bp, Artificial Sequence
  Description of Artificial Sequence Synthetic Primer
gccgggcgtcataaaaacagg

SEQ ID NO: 13 - DNA, 1799 bp, Brucella melitensis
caagcttcaggaaatggaagatcaggtcattcctgacatcattgcctgaacattcaagaacactatagggaagagccggggttcgcccggctttgtcttttcaagaccttcctcatgccaaccgaaaagccaagcaggaaagaccgatgagcgttgatgtcgccattatcatgggaagccagtccgattgggaaaccatgcaccatgcagccgacacattggaggcgctcggcatctccttcgacgcacggatcgtttccgcccatcgcacccctgacaggctggtcgccttcgccaagggggcgaaagcggaaggcttcaaggtcatcatcgcaggcgccggcggcgcggcccacctgcccggcatggccgctgccatgacaccgcttcccgtctttggcgttccagttcaatccaaggcgctttcgggccaggattcgcagatctaagcccggctccaccatcggcattatcggcgggggccagcttggccgtatgctcgccatggcagcggcgcgcttcggttatgaaaccataatccttgagccgcaggccggttgcccggcagcacaggttgccaatcgccagattgtcgccgcctatgatgacccgaaggcgctggccgaacttgccgccgcttccgacgtcatcacctatgaatttgaaaatgtgccagtcagcgccgccgacaagctggctgaaacggcgcttgttctgcccccgcccgccgcactggaaatctctcaggaccgcttcacagaaaagcagtttctcaacgaaagcggcattgaaaccgcgccctggcggctcgtggatgacgaggaaacgctcatcgccgcgctcggcgcactgggcgggcgtggcatcctcaagatacggcgtctgggttatgacggcaaggggcaggtgcgccttgcctccctcgatgaaacccaggcctgcaacgcttttgcagccatcaacaaggcgcctgcgattctcgaaggcttcgtggaattcgagcgcgaagtctccgtcatcgccgcgcgcgatcgcagcggcaatgtcgccatcttcgatcttgcggaaaacgtccacaaggatggcattctcgccacgtccacagtgcctgccgcgatcagcgtacagacggcagaagccgcgcgcacagccgccgaaaaactgttgcacgcgctggactatgtcggtgtgctggggcttgaattcttcgtgctgaaggacggcacgctgctcgccaatgaatttgccccgcgtgtgcataattcgggccactggacggaagcagcctgcgccatttcccaatttgagcagcatatccgcgctgtggcgggactgccgctcggcaatacggatcgccatagcgactgtgtgatggaaaacctgattggcgacgatatcgaaaaggttccggcgattctctgcgagaagaacgccgtgctgcatctttacggtaaaaaggaagctcgcgcgggccggaaaataggccatgtgacccgcataaagccccgcacaatttaagctgcgccgggaatctgcacgattcccggcccttcctggccgcccgccaagaaattcggggcctcggaccctgaatctgcggcgcgggagttgacatttgcctgaaaccttgtgtatttcggccaacccttcgggcacctgaccgtgcctgtaatcaattggcgccttgggcgcctgtttttatgacgcccggcggcatcttcatgtcgatgggccaaccagaccggtgattgacatgaagatcaagaactcgctcaaagccctcaaggcccgtcatcgcg

SEQ ID NO: 14 - DNA, 1633 bp, Brucella melitensis
aagctttttgcctatgccccgatcgagaacgtaccgccgggtgtcgaactgcatgatgccgaaagcattttgcctgaaacggcattcaagcggcttgatccggcctatccgaattttcatacccaactgactgttgttcaattcagtgatattttccgtatcatgctgattaaataccagcagggcgtctggctggatacggatgtctatcttctcaagcagtttcatcccgatccggacaagccatacctcgccagggagaatcgctcccgtgtcggtgtgtctgcgctttatcttccgcatgaccatccgattattcatgaattcgaggattacatggccgcgaccgatccgccgccgacctggctgggtctgcgcagaggcaaattgcgtccgctttactatcgattgatccacaaggaagtaacgcccgcctccattggcattacagtctttggcaatgacggtatctcacgcctcgcccgcaaatacgggattttcaaggatgccgcgccgcaggaaaatttctattactgggtcggcaaggaggccacgcgcatctatgatccggcctatggcctgacgccgatccatcatccggagtttatcggttttcatatccataaaaagcataaggaagtcgtgtcgttccagccgggaagtttttacatgtgggcaatagatcgggtgcgcccattgctggaatctaaagaaaacaaagagcttgcgctggcagattgaatcccgtggccttcgctgtatgtcaaatttgctacgagccaatgaaattgccggttgcgcaaagattcccctatttcgacaatcatgccgagccagcctgtacagatgtttgaaggcgaggaagcctgacgctttccgatatttgccggggcgtggtaagtatcccgccccagcttttgtttgagtatgtttcggccagaaagcgcatcgctatcgggcattgcttgaacgttccggggcgtcttcatttgaaacagcttacgcggtgatttatctgcaaggcagatccatgcgttcccgcgtccccggagaatattacttgtccaaacccaaggataaaaaagcggcatccctcgatggcagcaagggctttgccctttccgagcctaagccaacgagagcagccgtcattgttcccgtcctgccggaccgttataatagcggctctgcgggcgaggatggcgcaagatcgcagttccagcgttccaacgaggcgcgccttgaggaagcgatcgggcttgcgcgcgcaattggtcttgatatcgaacatgcggaaatcgccatcgtcaccaatccgcggcccgcgacgctgcttggtgcgggcaaggcccaatccattgccgaagtcgtcaaggaaaaggcgattggactggtggttgtcgatcatgccctgacgccggtgcagcagcgcaatcttgaaaaggaatggaacgccaaggtcatcgaccgaaccgggctcattctggagattttcggtgaacgcgcgcgcaccaaggaaggcgcgcttcaggtggagctggcgcatctcaactaccagaagggacgccttgtcagaagctggacccaccttgaacgccagcgcggcggtgggggcttccttggcggcccgggt

SEQ ID NO: 15 - DNA, 7448 bp, Brucella melitensis
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttgcctatgccccgatcgagaacgtaccgccgggtgtcgaactgcatgatgccgaaagcattttgcctgaaacggcattcaagcggcttgatccggcctatccgaattttcatacccaactgactgttgttcaattcagtgatattttccgtatcatgctgattaaataccagcagggcgtctggctggatacggatgtctatcttctcaagcagtttcatcccgatccggacaagccatacctcgccagggagaatcgctcccgtgtcggtgtgtctgcgctttatcttccgcatgaccatccgattattcatgaattcgaggattacatggccgcgaccgatccgccgccgacctggctgggtctgcgcagaggcaaattgcgtccgctttactatcgattgatccacaaggaagtaacgcccgcctccattggcattacagtctttggcaatgacggtatctcacgcctcgcccgcaaatacgggattttcaaggatgccgcgccgcaggaaaatttctattactgggtcggcaaggaggccacgcgcatctatgatccggcctatggcctgacgccgatccatcatccggagtttatcggttttcatatccataaaaagcataaggaagtcgtgtcgttccagccgggaagtttttacatgtgggcaatagatcgggtgcgcccattgctggaatctaaagaaaacaaagagcttgcgctggcagattgaatcccgtggccttcgctgtatgtcaaatttgctacgagccaatgaaattgccggttgcgcaaagattcccctatttcgacaatcatgccgagccagcctgtacagatgtttgaaggcgaggaagcctgacgctttccgatatttgccggggcgtggtaagtatcccgccccagcttttgtttgagtatgtttcggccagaaagcgcatcgctatcgggcattgcttgaacgttccggggcgtcttcatttgaaacagcttacgcggtgatttatctgcaaggcagatccatgcgttcccgcgtccccggagaatattacttgtccaaacccaaggataaaaaagcggcatccctcgatggcagcaagggctttgccctttccgagcctaagccaacgagagcagccgtcattgttcccgtcctgccggaccgttataatagcggctctgcgggcgaggatggcgcaagatcgcagttccagcgttccaacgaggcgcgccttgaggaagcgatcgggcttgcgcgcgcaattggtcttgatatcgaacatgcggaaatcgccatcgtcaccaatccgcggcccgcgacgctgcttggtgcgggcaaggcccaatccattgccgaagtcgtcaaggaaaaggcgattggactggtggttgtcgatcatgccctgacgccggtgcagcagcgcaatcttgaaaaggaatggaacgccaaggtcatcgaccgaaccgggctcattctggagattttcggtgaacgcgcgcgcaccaaggaaggcgcgcttcaggtggagctggcgcatctcaactaccagaagggacgccttgtcagaagctggacccaccttgaacgccagcgcggcggtgggggcttccttggcggcccgggtaccgagctcggatccactagtaacggccgccagtgtgctggaattcgcccttaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtatctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataaaaacgcaaaagaaaatgccgattatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtcatacactattatcccgtattgacgccgggcaagagcaactcggtcgccgggcgcggtattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 16 - DNA, 8931 bp, Brucella melitensis
ctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtaggctttacactttatgcttccggctcgtatgttgtgtggaattgtgagcggataacaatttcacacaggaaacagctatgaccatgattacgccaagcttcaggaaatggaagatcaggtcattcctgacatcattgcctgaacattcaagaacactatagggaagagccggggttcgcccggctttgtcttttcaagaccttcctcatgccaaccgaaaagccaagcaggaaagaccgatgagcgttgatgtcgccattatcatgggaagccagtccgattgggaaaccatgcaccatgcagccgacacattggaggcgctcggcatctccttcgacgcacggatcgtttccgcccatcgcacccctgacaggctggtcgccttcgccaagggggcgaaagcggaaggcttcaaggtcatcatcgcaggcgccggcggcgcggcccacctgcccggcatggccgctgccatgacaccgcttcccgtctttggcgttccagttcaatccaaggcgctttcgggccaggattcgcagatctaagcccggctccaccatcggcattatcggcgggggccagcttggccgtatgctcgccatggcagcggcgcgcttcggttatgaaaccataatccttgagccgcaggccggttgcccggcagcacaggttgccaatcgccagattgtcgccgcctatgatgacccgaaggcgctggccgaacttgccgccgcttccgacgtcatcacctatgaatttgaaaatgtgccagtcagcgccgccgacaagctggctgaaacggcgcttgttctgcccccgcccgccgcactggaaatctctcaggaccgcttcacagaaaagcagtttctcaacgaaagcggcattgaaaccgcgccctggcggctcgtggatgacgaggaaacgctcatcgccgcgctcggcgcactgggcgggcgtggcatcctcaagatacggcgtctgggttatgacggcaaggggcaggtgcgccttgcctccctcgatgaaacccaggcctgcaacgcttttgcagccatcaacaaggcgcctgcgattctcgaaggcttcgtggaattcgagcgcgaagtctccgtcatcgccgcgcgcgatcgcagcggcaatgtcgccatcttcgatcttgcggaaaacgtccacaaggatggcattctcgccacgtccacagtgcctgccgcgatcagcgtacagacggcagaagccgcgcgcacagccgccgaaaaactgttgcacgcgctggactatgtcggtgtgctggggcttgaattcttcgtgctgaaggacggcacgctgctcgccaatgaatttgccccgcgtgtgcataattcgggccactggacggaagcagcctgcgccatttcccaatttgagcagcatatccgcgctgtggcgggactgccgctcggcaatacggatcgccatagcgactgtgtgatggaaaacctgattggcgacgatatcgaaaaggttccggcgattctctgcgagaagaacgccgtgctgcatctttacggtaaaaaggaagctcgcgcgggccggaaaataggccatgtgacccgcataaagccccgcacaatttaagctgcgccgggaatctgcacgattcccggcccttcctggccgcccgccaagaaattcggggcctcggaccctgaatctgcggcgcgggagttgacatttgcctgaaaccttgtgtatttcggccaacccttcgggcacctgaccgtgcctgtaatcaattggcgccttgggcgcctgtttttatgacgcccggcggcatcttcatgtcgatgggccaaccagaccggtgattgacatgaagatcaagaactcgctcaaagccctcaaggcccgtcatcgcgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagcgggaattaattctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagttcgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaccgccctgcaaggcggttttttcgttttcagagcaagagattacgcgcagaccaaaacgatctcaagaagatcatcttattaatcagataaaatatttctagctagagtcgatcttcgccagcagggcaggatcgtggcatcaccgaaccgcgccgtgccgctcgtcggtgagccagagtttcagcaggccgcccagggggaggtcgccattgatgcgggccagctcgcggacgtgctcatagtccacgacgcccgtgattttgtagccctggccgacgcgacaggtaggccgacagctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacgcgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatcgactctagctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcgcggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcatttcgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtggtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcactaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatgaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccaagctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgggtttgcaactgcgggtcaaggtctggatttcgatcacggcacgatcatgctgcgggagggcaagggctccaaggatcgggccttgatgttacccgagagcttggcacccacgctgcgcgagcagaattaattccc